(19) United States
(12) Patent Application Publication
(10) Pub. No.: US 2010/0291089 A1
(43) Pub. Date: Nov. 18, 2010

(54) LIGAND OF REGULATING IMMUNE RESPONSE, AND USE THEREOF IN TREATING AN IMMUNE RESPONSE-RELATED DISEASE

(75) Inventors: Youn-Dong Kim, Pohang-city (KR); Byoung-Dae Lee, Pohang-city (KR); Yoe-Kyung Kim, Pohang-city (KR); Yoe-Sik Bae, Busan-city (KR); Tae-Hoon Lee, Pohang-city (KR); Pann-Ghill Suh, Pohang-city (KR); Sung-Ho Ryu, Pohang-city (KR)

Correspondence Address: SHERIDAN ROSS PC, 1560 BROADWAY, SUITE 1200, DENVER, CO, 80202, US

(73) Assignee: POSTECH ACADEMY-INDUSTRY FOUNDATION, Pohang-city (KR)

(21) Appl. No.: 11/993,655

(22) PCT Filed: Jul. 7, 2006
(86) PCT No.: PCT/KR2006/002659

§ 371 Date: Dec. 21, 2007

Related U.S. Application Data
(60) Provisional application No. 60/595,458, filed on Jul. 7, 2005.

(51) Int. Cl. A61K 39/395; C07K 14/435; A61K 38/08; A61K 39/10; A61K 31/5377; A61K 31/352; A61K 31/713; A61P 37/02; A61P 29/00; A61P 25/28; A61P 9/10; A61P 35/00

(52) U.S. Cl. 424/139.1; 530/350; 514/20.6; 424/240.1; 514/233.5; 514/456; 514/44 A

(57) ABSTRACT

The present invention relates to a ligand to regulate immune response, i.e., PACAP27 which is one of pituitary adenylate cyclase-activating polypeptides and Serum amyloid A (SAA), and their novel use in treating or preventing diseases associated with immune response. More specifically, the present invention relates to a complex of PACAP27-FPRL1 having a regulatory effect on immune response, and a use thereof in regulating immune response. In another aspect, the present invention relates to a complex of SAA and FPRL1, and a use thereof in inhibiting synoviocyte hyperplasia and angiogenesis and treating or preventing inflammatory diseases including Rheumatoid arthritis (RA).

### CROSS REFERENCE TO RELATED APPLICATION

This application is a National Phase 35 U.S.C. 371 filing of PCT/KR2006/002659, filed Jul. 7, 2006; which claims priority to and the benefit of U.S. Patent Provisional Application No. 60/595,458 filed on Jul. 7, 2005; which are hereby incorporated by reference for all purposes as if fully set forth herein.

Incorporated by reference herein in its entirety is the Sequence Listing, entitled “5413PAF-1_ST25.txt”, created May 17, 1010, size of 12 kilobytes.

### BACKGROUND OF THE INVENTION

(a) Field of the Invention

The present invention relates to a ligand to regulate immune response, i.e., PACAP27 (SEQ ID NO:1) which is one of pituitary adenylate cyclase-activating polypeptides and Serum amyloid A (SAA) (SEQ ID NO:19), and their novel use in treating or preventing diseases associated with immune response. More specifically, the present invention relates to a complex of PACAP27-FPRL1 having a regulatory effect on immune response, and a use thereof in regulating immune response. In another aspect, the present invention relates to a complex of SAA (SEQ ID NO:19) and FPRL1 (SEQ ID NO:4), and a use thereof in inhibiting synoviocyte hyperplasia and angiogenesis and treating or preventing inflammatory diseases including Rheumatoid arthritis (RA).

(b) Description of the Related Art

The two pituitary adenylate cyclase-activating polypeptides (PACAPs), PACAP27 (SEQ ID NO:1) and PACAP38 (SEQ ID NO:2), are neuropeptides that belong to the secretin/glucagon/vasoactive intestinal peptide (VIP) (SEQ ID NO:3) family. PACAPs are multifunctional peptide hormones that influence diverse biological functions, e.g., the cell cycle, smooth muscle and cardiac muscle relaxation, bone metabolism, and endocrine/paracrine function. In addition, during recent years, the effects of PACAPs on the immune system have been partially elucidated. In this context, both of PACAPs suppress or activate inflammation by regulating the interleukins, IL-1, IL-6, and IL-10.

Three distinct G-protein coupled receptors (GPCR) of PACAPs have been identified as PAC1, VPAC1 and VPAC2. PAC1 can be activated by PACAPs, but not by VIP (SEQ ID NO:3), whereas VPAC1 and VPAC2 are activated by both. PAC1 has been reported to inhibit IL-6 production in stimulated macrophages, despite its up-regulation of IL-6 secretion in unstimulated macrophages. However, the specific nature of the involvement of PACAP receptors in immune-related functions has yet to be adequately demonstrated. Therefore, in treating the diseases associated with immune response and developing new drugs therefore, it has been required to elucidate PACAP-mediated immune cell functions by investigating the receptor expression pattern.

Meanwhile, rheumatoid arthritis (RA) is a multi-system autoimmune disease, which is characterized by chronic joint inflammation. The hallmark characteristics of RA pathology include the infiltration of inflammatory leukocytes, the proliferation of synovial cells, and the presence of extensive angiogenesis, which is also commonly referred to as rheumatoid pannus. Rheumatoid pannus is sometimes considered to be a local tumor. For example, synovial fibroblasts, the principal components of invading pannus, proliferate abnormally, resist apoptosis, and invade the local environment. Synovial fibroblasts obtained from RA patients exhibit several oncogenes, including H-ras and p53, harboring somatic mutations. They also abundantly express anti-apoptotic proteins, including the FLICE inhibitory protein (FLIP) and Bcl-2, both of which exert protective effects against the apoptosis initiated via death receptor- or mitochondria-dependent pathways. Moreover, in a fashion similar to that of carcinogenesis, angiogenesis is considered to be a critical step in the progression of RA.

Serum amyloid A (SAA; SEQ ID NO:19) is a multi-functional apolipoprotein, 12- to 14-kDa in size. This protein is normally present in the bloodstream at a concentration of approximately 0.1 μM, but the concentration of SAA (SEQ ID NO:19) can increase up to 1000-fold within the first 24 to 36 h in response to a variety of injuries, including trauma, infection, inflammation, and neoplasia. As with other acute-phase reactants, the liver is the primary site at which SAA (SEQ ID NO:19) production occurs, but the overproduction of SAA (SEQ ID NO:19) in extrahepatic areas has also been implicated in the pathogenesis of several chronic inflammatory diseases, including human atherosclerosis, Alzheimer's disease, inflammatory arthritis, and several cancer variants. Moreover, elevated SAA (SEQ ID NO:19) levels appear to be an important indicator for both the diagnosis and prognosis of certain inflammatory diseases. For example, increased levels of SAA (SEQ ID NO:19) are frequently observed in the sera, synovial fluid, and inflamed synovium of RA patients, and these levels have been commonly used as highly sensitive markers for the disease activity of RA.

There are two known SAA receptors, including CD36 and LIMPII analoguous-1 (CLA-1), and lipoxin A4 receptor (LXA4R)/formyl peptide receptor like 1 (FPRL1; SEQ ID NO:4). FPRL1 (SEQ ID NO:4) is one of the classic chemoattractant receptors encompassing G protein-coupled seven transmembrane domains. Previous reports have pointed to a role for FPRL1 (SEQ ID NO:4) in the regulation of a variety of cellular responses in several cell types, including astrocytoma cell lines (24), neutrophils, monocytes, and T cells (25), and human umbilical vein endothelial cells (HUVECs) (26). Recently, O'Hara et al. showed that overexpressed SAA (SEQ ID NO:19) and FPRL1 (SEQ ID NO:4) in inflamed synovial tissue can be associated with the production of matrix metalloproteinase (MMP) (27). However, it remains to be determined whether SAA (SEQ ID NO:19) and FPRL1 (SEQ ID NO:4) in the RA synovium are involved directly in the synovial proliferation and formation of an invading pannus. Furthermore, very little information is currently available regarding the intracellular pathway relevant to SAA signaling in RA synoviocytes.

### SUMMARY OF THE INVENTION

The object of the present invention is to provide a complex of PACAP27-FPRL1 having a regulatory effect on immune response.

Another object of the present invention is to provide a composition of treating or preventing diseases associated with immune response including inflammatory diseases, containing an inhibitor to inactivate the activity of PACAP27 and/or FPRL1, or inhibit the binding of PACAP27 (SEQ ID NO:1) to FPRL1 (SEQ ID NO:4).

Another object of the present invention is to provide a method of treating or preventing diseases associated with immune response including inflammatory diseases by inactivating the activity of PACAP27 and/or FPRL1, or inhibiting the binding of PACAP27 (SEQ ID NO:1) to FPRL1 (SEQ ID NO:4) to inhibit the formation of the PACAP27-FPRL1 complex.

Another object of the present invention is to provide a target for developing drugs treating or preventing diseases associated with immune response including inflammatory diseases containing the PACAP27-FPRL1 complex.

Another object of the present invention is to provide a complex of SAA-FPRL1 having a regulatory effect on immune response.

Another object of the present invention is to provide a composition of treating or preventing inflammatory diseases including Rheumatoid arthritis (RA), containing an inhibitor to inactivate the activity of SAA and/or FPRL1, or inhibit the binding of SAA (SEQ ID NO:19) to FPRL1 (SEQ ID NO:4), wherein the composition has an inhibitory effect of synoviocyte hyperplasia and angiogenesis.

Another object of the present invention is to provide a method of inhibiting synoviocyte hyperplasia and angiogenesis by inactivating the activity of SAA and/or FPRL1, or inhibiting the binding of SAA (SEQ ID NO:19) to FPRL1 (SEQ ID NO:4) to inhibit the formation of the SAA-FPRL1 complex.

Another object of the present invention is to provide a method of treating or preventing inflammatory diseases including RA by inactivating the activity of SAA and/or FPRL1, or inhibiting the binding of SAA (SEQ ID NO:19) to FPRL1 (SEQ ID NO:4) to inhibit the formation of the SAA-FPRL1 complex.

Still another object of the present invention is to provide a target for developing drugs treating or preventing inflammatory diseases including RA containing complex of SAA (SEQ ID NO:19) and FPRL1 (SEQ ID NO:4).

### BRIEF DESCRIPTION OF THE DRAWINGS

FIGS. 1A, 1B and 1C show that PACAP27 (SEQ ID NO:1) selectively induces intracellular signaling in human neutrophils. In 1A and 1B, fura-2-loaded human neutrophils were treated with agonist peptides (PACAP27 (SEQ ID NO:1), PACAP38 (SEQ ID NO:2), or VIP (SEQ ID NO:3)). Changes at 340 nm and 380 nm were monitored and fluorescence ratios were converted to [Ca²⁺]ᵢ. Neutrophils were stimulated with 1 μM of PACAP27 (SEQ ID NO:1), PACAP38 (SEQ ID NO:2), or VIP (SEQ ID NO:3) (1A). Dose dependency was tested at various concentrations (50 nM to 5 μM) of PACAP27 (SEQ ID NO:1), PACAP38 (SEQ ID NO:2), or VIP (SEQ ID NO:3) in human neutrophils (2B). Data are presented as means±SE of four independent experiments, each of which was performed in triplicate (2B). ERK phosphorylation was assessed by Western blotting, using a phospho-ERK-specific antibody. Neutrophils were incubated with various concentrations (200 nM to 20 μM) of PACAP27 (SEQ ID NO:1), PACAP38 (SEQ ID NO:2), or VIP (SEQ ID NO:3) (1C). The results shown are representative of four independent experiments, each performed in duplicate.

FIGS. 2A, 2B, 2C, 2D, 2E, 2F, 2G, and 2H show that PACAP27 (SEQ ID NO:1) activates human neutrophils via FPRL1 (SEQ ID NO:4). In 2A-2E, and 2H, fura-2-loaded human neutrophils or RBL-2H3 cells were treated with agonist peptides. Changes at 340 nm and 380 nm were monitored and fluorescence ratios were converted to [Ca²⁺]ᵢ. Neutrophils were treated with 2 μg/ml PTX (2A) or 2 μM U73122 (2B) prior to being stimulated with PACAP27 (SEQ ID NO:1), for 3 hours or 30 minutes, respectively. In 2C, neutrophils were stimulated with 1 μM of PACAP27 (SEQ ID NO:1) and this was followed by adding 10 nM WKYMVm (SEQ ID NO:5) (i), or 10 nM WKYMVm (SEQ ID NO:5) and then 1 μM of PACAP27 (SEQ ID NO:1) (ii). In 2D, FPRL1/RBL, FPR/RBL, and vector/RBL cells were stimulated with 1 μM of PACAP27 (SEQ ID NO:1), PACAP38 (SEQ ID NO:2), or VIP (SEQ ID NO:3). In 2E, neutrophils were stimulated with vehicle or with various WRW4 (SEQ ID NO:6) concentrations for 30 seconds prior to the addition of 1 μM PACAP27 (SEQ ID NO:1). The results shown are representative of four independent experiments performed in duplicate. In 2F, ERK phosphorylation was assessed by Western blotting using phospho-ERK specific antibody. Neutrophils were incubated with either vehicle or 1 μM WRW4 (SEQ ID NO:6) for 30 seconds and then treated for 5 minutes with 10 nM WKYMVm (SEQ ID NO:5) or 1 μM PACAP27 (SEQ ID NO:1). Data present the means±SE of four independent experiments performed in triplicate (2D, and 2E). In 2G, cAMP elevation was measured, as described in “Materials and Methods”. Neutrophils were stimulated with vehicle or with 1 μM WRW4 (SEQ ID NO:6) for 30 seconds and then treated with 10 μM PACAP27 (SEQ ID NO:1) for 10 minutes. In 2H, Human monocytes were treated with vehicle or with 1 μM WRW4 (SEQ ID NO:6) for 30 seconds prior to the addition of various PACAP27 (SEQ ID NO:1) concentrations. The results shown are representative of four independent experiments performed in duplicate. *, p<0.01 vs vehicle treatment.

FIG. 3 shows that PACAP27 (SEQ ID NO:1) primes fMLP-induced calcium signaling via FPRL1 (SEQ ID NO:4) dependent. Changes at 340 nm and 380 nm were monitored and fluorescence ratios were converted to [Ca²⁺]ᵢ. Neutrophils were treated with vehicle or 1 μM WRW4 (SEQ ID NO:6) for 30 seconds, prior to being stimulated with vehicle, 1 μM PACAP27 (SEQ ID NO:1), 10 nM fMLP, or both. The results shown are representative of two independent experiments performed in duplicate. *, p<0.01 vs control.

FIGS. 4A, 4B, 4C, and 4D show that PACAP27 (SEQ ID NO:1) induces the up-regulation of CD11b in neutrophils via FPRL1 (SEQ ID NO:4). Surface CD11b expression was determined via FACS analysis. Neutrophils were gated out (4A); CD11b-levels are represented by mean fluorescence intensity (4B) or histograms (4C). Purified neutrophils were incubated with various concentrations of PACAP27 (SEQ ID NO:1) for 1 hour (4B) or with vehicle or 1 μM WRW4 (SEQ ID NO:6) for 30 seconds prior to being treated with 10 μM PACAP27 (SEQ ID NO:1) for 1 hour (4C). D, Purified neutrophils were incubated with 1 μM or 10 μM of PACAP27 (SEQ ID NO:1), heat-inactivated PACAP27 (SEQ ID NO:1), or polymyxin b-treated PACAP27 (SEQ ID NO:1) for 1 hour. Heat-inactivation was performed for 10 minutes in boiling water. PACAP27 (SEQ ID NO:1) was pretreated with 5 μM polymyxin b for 1 hour in 37° C. The results shown are representative of four independent experiments performed in duplicate.

FIGS. 5A and 5B show that PACAP27 (SEQ ID NO:1) induces neutrophil chemotaxis via FPRL1 (SEQ ID NO:4). Chemotaxis assays were conducted using a modified Boyden chamber assay, as described in “Materials and Methods”. Neutrophil chemotaxis was examined using various concentrations of PACAP27 (SEQ ID NO:1) (5A). Neutrophils were tested using vehicle, 10 nM WKYMVm (SEQ ID NO:5), or 1 μM PACAP27 (SEQ ID NO:1) in the absence and presence of 1 μM WRW4 (SEQ ID NO:6) (5B). Data are presented as the means±SE for migrated neutrophils per field were counted in triplicate of four independent experiments. *, p<0.01 vs vehicle treatment.

FIGS. 6A, 6B, 6C, 6D, and 6E show that the FPRL1-PACAP27 interaction is mediated predominantly by the C-terminal region of PACAP27 (SEQ ID NO:1). Truncated PACAPs (tPACAP) and chimeric PACAPs (cPACAP) were tested using FPRL1 (SEQ ID NO:4)-expressing RBL-2H3 cells. EC₅₀ values were obtained by measuring increases in [Ca²⁺]ᵢ activity (6A). FPRL1/RBL cells (1×10⁵ cells/200 μL) were used for the binding assay (6B). FPRL1/RBL cells were pretreated with various concentrations of unlabeled PACAP27 (SEQ ID NO:1) or tPACAPs prior to being treated with ¹²⁵I-labeled PACAP27 (SEQ ID NO:1) (50 μM). Controls were prepared by pretreating with vehicle prior to ¹²⁵I-labeled PACAP27 (SEQ ID NO:1) treatment (6B). The amino acid sequences of PACAP27 (SEQ ID NO:1) and VIP (SEQ ID NO:3) were compared, and 4 residues were selected (▾) for the construction of chimeras on the basis of their chemical properties (6C). The EC₅₀ values of cPACAPs with respect to increasing [Ca²⁺]ᵢ activity were measured (6D). The receptor binding affinities of the cPACAPs were determined in a manner identical to that used for tPACAPs (6E). Data are presented as means±SE of four independent experiments performed in triplicate (6A-6B, 6D-6E).

FIGS. 7A, 7B, and 7C show that proliferative effect of SAA (SEQ ID NO:19) on FLS. RA FLS and OA FLS were treated with increasing concentrations of SAA (SEQ ID NO:19) (0-5 μM) for 72 h. Primary cultured RA FLS and OA FLS were plated in triplicate, and [³H] thymidine incorporation was employed in the measurement of DNA synthesis activity in the presence of SAA (SEQ ID NO:19) (0, 0.1, 1, 3, or 5 μM) for 72 h (7A). After 72 h of incubation with increasing doses of SAA (SEQ ID NO:19) (0, 0.1, 1, 3, or 5 μM), the RA FLS and OA FLS were trypsinized, and the cell numbers per well were determined under a microscope (7B). RA FLS incubated in the presence or absence of 5 μM SAA (SEQ ID NO:19) for 72 h were photographed (7C). Original magnification, ×50. The results are presented as the mean±SD of three independent experiments using different cells.

FIGS. 8A, 8B, 8C, and 8D show that increased viability of RA FLS by SAA (SEQ ID NO:19) treatment. In the MTT assay (8A) and cellular DNA fragmentation assay (8B), RA FLS and OA FLS were treated with increasing concentrations of SAA (SEQ ID NO:19) (0-5 μM) under serum-deprivation conditions for 72 h. The levels of cellular DNA fragmentation of RA FLS induced by sodium nitroprusside (SNP, 0.7 mM) or IgM anti-FAS Ab (0.7 μg/ml) plus cycloheximide (CHX, 1.0 μg/ml) were measured in either the presence or absence of SAA (SEQ ID NO:19) (3 μM) for 12 h (8C). Representative phase-contrast microscopy of RA FLS apoptosis was conducted 12 h after SNP treatment (0.7 mM) in the presence or absence of SAA (SEQ ID NO:19) (3 μM) (8D). Original magnification: ×50. Data are presented as mean±SD of three independent experiments.

FIGS. 9A, 9B, 9C, and 9D show that increased proliferation and survival of RA FLS by SAA (SEQ ID NO:19) via FPRL1 (SEQ ID NO:4). FPRL1 expression levels in RA FLS and OA FLS cultured from five RA and five OA patients, respectively, was analyzed via RT-PCR (9A). The specific agonist for FPRL1 (SEQ ID NO:4), WKYMVm (SEQ ID NO:5) peptide was added to the RA FLS and OA FLS in a concentration range of 1 to 100 nM. After 72 h, the proliferative effects of WKYMVm (SEQ ID NO:5) were evaluated via a [³H]-thymidine incorporation assay, and the survival activity of WKYMVm (SEQ ID NO:5) was determined via a MTT assay (9B). The downregulation of FPRL1 mRNA by short interfering RNA (siRNA) was established, and the mRNA expression levels for FPRL1 were determined via RT-PCR. BL (Blank); no addition of siRNA, CO (Control); luciferase siRNA, F1; FPRL1 siRNA (target probe: 300-320), F2; FPRL1 siRNA (target probe: 403-423) (9C). After 48 h, the incubation of FPRL1 knock-downed MH7A cells in the presence or absence of SAA (5 μM), DNA synthesis (upper panel) and apoptosis (lower panel) were conducted via [³H]-thymidine incorporation assay and DNA fragmentation ELISA. BL (Blank); no addition of siRNA, CO (Control); luciferase siRNA, F1; FPRL1 siRNA (target probe: 300-320), F2; FPRL1 siRNA (target probe: 403-423) (9D). Data are presented as mean±SD of four independent experiments with similar results.

FIGS. 10A and 10B show that SAA-induced increases in intracellular Ca²⁺ levels. Fluo-3 AM-loaded RA FLS and OA FLS were stimulated with SAA (SEQ ID NO:19) (3 μM) (10A) and WKYMVm (SEQ ID NO:5) (10 nM), an agonistic peptide for FPRL1 (SEQ ID NO:4) (10B), after which the relative levels of intracellular Ca²⁺ were monitored with a calcium-imaging system. In panel B, pertussis toxin (PTX) (100 ng/ml) pretreatment was administered to FLS for 12 h prior to the addition of WKYMVm (SEQ ID NO:5). The results are presented as the mean±SD of three independent experiments using different cells.

FIGS. 11A, 11B, and 11C show that activation of intracellular signaling molecules by SAA (SEQ ID NO:19) in RA FLS. RA FLS were incubated with 3 μM SAA (SEQ ID NO:19) (11A) and 10 nM WKYMVm (SEQ ID NO:5) (11B) for the indicated times, and ERK, Akt, and STAT3 phosphorylation were determined via Western blot analysis (upper panel of A and B). ERK and Akt activation were assessed after the application of treatment with the indicated amounts of SAA (SEQ ID NO:19) for 5 minutes and WKYMVm (SEQ ID NO:5) for 10 minutes (middle panel of A and B). RA FLS stimulated with SAA (SEQ ID NO:19) (3 μM) and WKYMVm (SEQ ID NO:5) (10 nM) for various times were lysed, subjected to Western blot analysis, and then evaluated using anti-cyclin D1 or anti-Bcl-2 antibodies. Actin was used for the verification of equal protein loading in each lane (lower panels of A and B). RA FLS were pretreated with Pertussis toxin (100 ng/ml, PTX), U73122 (1 μM), PD98059 (50 μM), or LY294002 (50 μM) prior to the addition of SAA (SEQ ID NO:19) (5 μM) (11C). After 72 h of incubation, the DNA synthesis (upper panel) and survival (lower panel) characteristics of the RA FLS were assessed via a [³H]-thymidine incorporation assay and an MTT assay, respectively. Data are presented as mean±SD of three independent experiments with similar results.

FIGS. 12A, 12B, 12C, 12D, 12E, 12F, 12G, and 12H show that effects of SAA on in vitro, ex vivo, and in vivo angiogenesis. The angiogenesis assays were conducted as described in the “Materials and Methods” section. The HUVECs were plated on M199 supplemented with 20% serum. After 12 h of culture, different doses of SAA (SEQ ID NO:19) (0-5 μM) (12A), WKYMVm (SEQ ID NO:5) (10 nM), or VEGF (20 ng/ml) were added to M199 medium supplemented with 1% serum. At 48 h, the amounts of DNA amount were determined via quantitation of the incorporated thymidine. Wm; WKYMVm (SEQ ID NO:5), VE; VEGF (12B): Confluent HUVECs were wounded with the tip of a micropipette, and incubated further in M199 containing 1% serum with SAA (SEQ ID NO:19) (0-5 μM), WKYMVm (SEQ ID NO:5) (10 nM), or VEGF (20 ng/ml). After 12 h, the cells migrating beyond the reference line were photographed (×50) and counted. Wm; WKYMVm (SEQ ID NO:5), VE; VEGF (12C): The HUVECs were seeded on 48 wells pre-coated with Matrigel, and incubated in the presence of SAA (SEQ ID NO:19) (5 μM), WKYMVm (SEQ ID NO:5) (10 nM), or VEGF (20 ng/ml) for 18 h (×50). The bar graph shows the total length of the tubes formed by the HUVECs. Wm; WKYMVm (SEQ ID NO:5), VE; VEGF (12D): Rat aortic explants were incubated in M199 harboring different dosages of SAA (SEQ ID NO:19) (3 and 5 μM), WKYMVm (SEQ ID NO:5) (100 nM), VEGF (20 ng/ml) or 10% FBS. After 7 days, the ECs sprouting from the explants were photographed. Three independent experiments were then conducted, each in duplicate. Wm; WKYMVm (SEQ ID NO:5) (12E-12H): C57BL/6 mice were injected s.c. with 0.5 ml of Matrigel supplemented with PBS, SAA (SEQ ID NO:19) (80 μg), or WKYMVm (SEQ ID NO:5) (1 μg). After 7 days, the mice were sacrificed and the matrigel plugs were excised and fixed. (E) Representative Matrigel plugs containing PBS, SAA (SEQ ID NO:19) (80 μg), or WKYMVm (SEQ ID NO:5) (1 μg), and the quantification of neovessel formation via measurements of the hemoglobin within the Matrigels. Wm; WKYMVm (SEQ ID NO:5), bars, ±SD. Statistical comparisons were conducted via Student's t-tests. *P<0.05 versus the hemoglobin contents of the Matrigel containing PBS. Representative photograph of the gels shown in cross-section, and stained with H&E (12F-12H). Magnification: ×100. Seven mice were used. Each of the values represents the mean from at least five animals, and similar results were obtained with two different experiments.

### DETAILED DESCRIPTION OF THE PREFERRED EMBODIMENTS

A more complete appreciation of the invention, and many of the attendant advantages thereof, will be readily apparent as the same becomes better understood by reference to the following detailed description.

The present invention provides a complex of PACAP27-FPRL1 having a regulatory effect on immune response. Further, the present invention provides a composition of treating or preventing diseases associated with immune response including inflammatory diseases, containing an effective amount of an inhibitor to inactivate the activity of PACAP27 (SEQ ID NO:1) and/or FPRL1 (SEQ ID NO:4), or to inhibit the binding of PACAP27 (SEQ ID NO:1) to FPRL1 (SEQ ID NO:4) to inhibit the formation of the PACAP27-FPRL1 complex. Further, the present invention is to provide a method of treating or preventing diseases associated with immune response including inflammatory diseases by inactivating the activity of PACAP27 (SEQ ID NO:1) and/or FPRL1 (SEQ ID NO:4), or inhibiting the binding of PACAP27 (SEQ ID NO:1) to FPRL1 (SEQ ID NO:4) to inhibit the formation of the PACAP27-FPRL1 complex. Furthermore, the present invention is to provide a target for developing drugs treating or preventing diseases associated with immune response including inflammatory diseases containing the PACAP27-FPRL1 complex. The diseases include, but not limited to, atherosclerosis, Alzheimer's disease, cancer, and rheumatoid arthritis (RA).

In the present invention, human PACAP (NCBI accession no. P18509; SEQ ID NO:20) and human FPRL1 (NCBI accession no. P25090; SEQ ID NO:4) are employed. However, the amino acid sequences of PACAP (SEQ ID NO:20) and FPRL1 (SEQ ID NO:4) are well conserved between different species, and thus, the present invention may be applied to all animals including the human being.

Although the neuropeptide pituitary adenylate cyclase activating polypeptide (PACAP; SEQ ID NO:20) has been implicated in the regulation of several immune responses, its target receptors and signaling mechanisms have yet to be fully elucidated in immune cells. In the present invention, it is found that PACAP27 (SEQ ID NO:1; 27 amino acids), but not PACAP38 (SEQ ID NO:2; 38 amino acids), wherein 27 amino acids of N-terminus are identical to those of PACAP27 (SEQ ID NO:1)), specifically stimulates intracellular calcium mobilization and extracellular signal-regulated kinase (ERK) phosphorylation in human neutrophils. Moreover, formyl peptide receptor-like 1 (FPRL1; SEQ ID NO:4) is identified as a PACAP27 (SEQ ID NO:1) receptor, and PACAP27 (SEQ ID NO:1) is found to selectively stimulate intracellular calcium increase in FPRL1-transfected rat basophile leukocytes (RBL)-2H3 cell lines. In addition, PACAP27-induced calcium increase and extracellular signal-regulated kinase phosphorylation are specifically inhibited by an FPRL1 (SEQ ID NO:4) antagonist, Trp-Arg-Trp-Trp-Trp-Trp (WRW4; SEQ ID NO:6), thus supporting the notion that PACAP27 (SEQ ID NO:1) acts on FPRL1 (SEQ ID NO:4). In terms of the functional role of PACAP27 (SEQ ID NO:1), it is found that the peptide stimulates CD11b surface up-regulation and neutrophil chemotactic migration, and that these responses are completely inhibited by WRW4 (SEQ ID NO:6). The interaction between PACAP27 (SEQ ID NO:1) and FPRL1 (SEQ ID NO:4) is analyzed further using truncated PACAPs and chimeric PACAPs using vasoactive intestinal peptide (VIP; SEQ ID NO:3), and the C-terminal region of PACAP27 (SEQ ID NO:1) is found to perform a vital function in the activation of FPRL1 (SEQ ID NO:4). Taken together, it may be suggested that PACAP27 (SEQ ID NO:1) activates phagocytes via FPRL1 (SEQ ID NO:4) activation, and that this results in pro-inflammatory behavior, involving chemotaxis and the up-regulation of CD11b.

The present inventors undertook to elucidate PACAP-mediated immune cell functions by investigating the receptor expression pattern, to complete the present invention. In the present invention, the functional roles of PACAP in human neutrophils, a type of phagocytic leukocyte are characterized, and the cell surface receptors involved in these processes are identified. Interestingly, it is found that PACAP27 (SEQ ID NO:1) exerts a stimulatory effect on an important chemoattractant receptor, formyl peptide receptor-like 1 (FPRL1; SEQ ID NO:4). In addition, an analysis of the region of PACAP27 (SEQ ID NO:1) found crucial for the binding and activation of FPRL1 (SEQ ID NO:4), its specific receptor, is conducted.

In the present invention, PACAP27-specific signaling in human neutrophils and its relations with calcium and ERK signaling, the up-regulation of CD11b, and with chemotactic migration may be observed. Previously known receptors like PAC1, VPAC1, and VPAC2 were found to be unhelpful in terms of explaining these PACAP27-specific activities, and thus, the present inventors hypothesized that another receptor is involved in this process. The present invention reveals that this receptor is FPRL1 (SEQ ID NO:4).

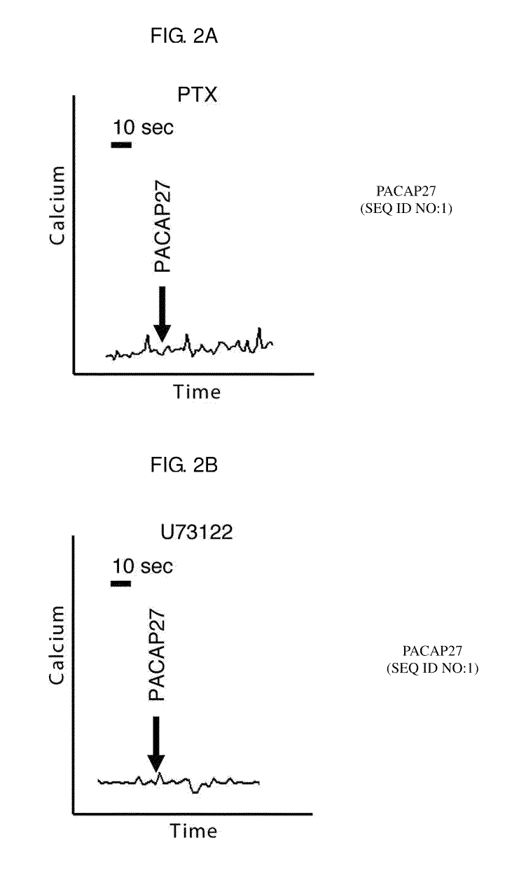

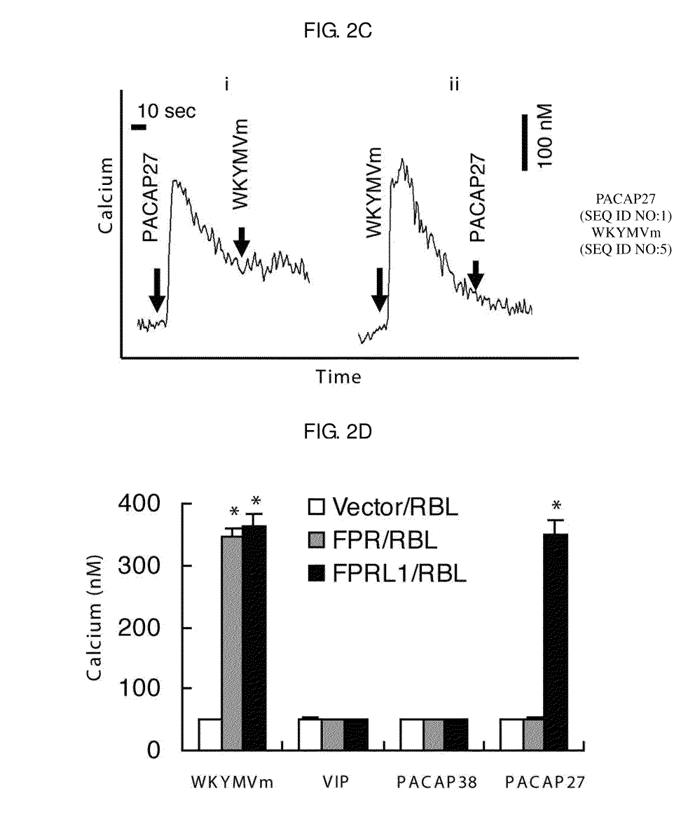

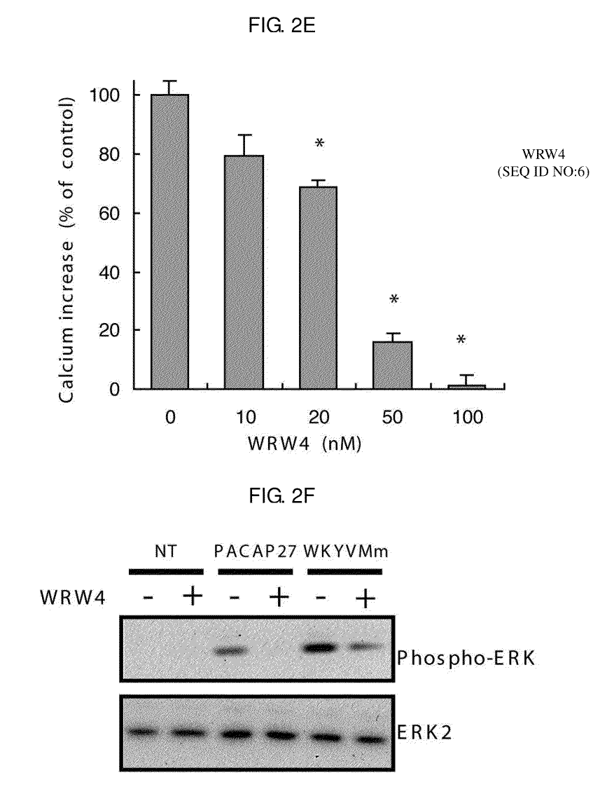

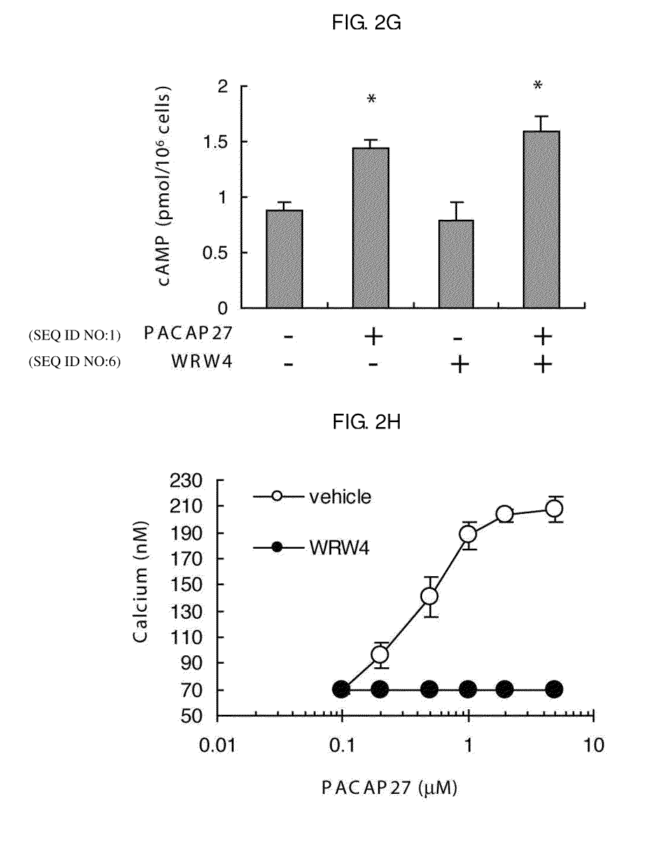

In order to prove the hypothesis that another receptor is involved in the PACAP27-specific activities, the cross-desensitization between PACAP27 (SEQ ID NO:1) and WKYMVm (SEQ ID NO:5) is revealed (see FIG. 2B). Cross-desensitization provides a straightforward and powerful means of illustrating receptor sharing. However, some GPCR groups do co-desensitize via single receptor activation for reasons, like receptor oligomerization, sequestration, and others. In order to solve this problem, in the present invention, the antagonizing peptide, WRW4 (SEQ ID NO:6), which does not activate but does bind FPRL1 (SEQ ID NO:4), is utilized. Desensitization events between two GPCRs usually occur via agonist-induced receptor activation. In addition, it is shown that the FPRL1-specific antagonist peptide, WRW4 (SEQ ID NO:6), can inhibit PACAP27 induced calcium signaling. FPRL1-expressing RBL2H3 cells are used to confirm this effect, and it was found that PACAP27-specific signaling only occurred on FPRL1-expressing cells (see FIG. 2D). These findings indicated that PACAP27 (SEQ ID NO:1) specifically activates human neutrophils by activating FPRL1 (SEQ ID NO:4).

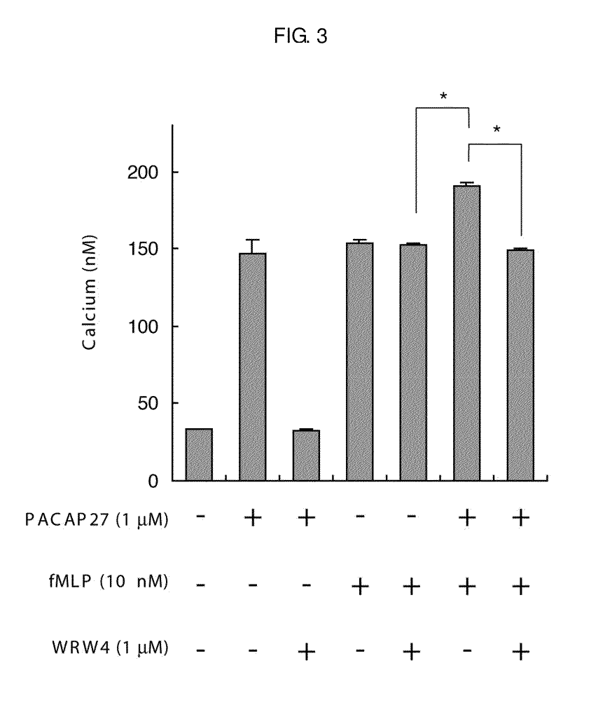

Previously, it has been reported that PACAP27 (SEQ ID NO:1) primes neutrophil response to the fMLP. Bacterial fMLP can activate, and FPRL1 (SEQ ID NO:4), at high concentrations, but fMLP activates only FPR at low concentrations. Therefore, in the present invention, it is hypothesized that the PACAP27-induced priming event on fMLP signaling is a result of the combined activation of these two receptors, FPRL1 (SEQ ID NO:4) and FPR. In order to prove this hypothesis, the effect of WRW4 (SEQ ID NO:6) on priming event is measured (see FIG. 3), and found that this event is FPRL1 (SEQ ID NO:4) dependent.

The regulation of the immune system by PACAP (SEQ ID NO:20) is likely to occur in a complex manner, as reflected by the inflammatory cytokine secretions of several immune cells. In monocytes and macrophages, PACAP molecules suppress the production of the pro-inflammatory cytokines, TNF-α, IL-6, and IL-12. On the other hand, in unstimulated macrophages and astrocytes, PACAP molecules initiate the IL-6 secretion, which induces a pro-inflammatory response. Chemotactic migration events also show this degree of complexity. PACAPs have a stimulatory effect on macrophage chemotaxis, but an inhibitory effect on lymphocyte chemotaxis, suggesting that PACAP can both promote and inhibit immune response. Although PACAP functioning has been examined by analyzing the expression patterns of various specific receptors (e.g., PAC1, VPAC1, and VPAC2), no evidence sufficiently explains this complexity. However, in the present invention, it is shown for the first time that FPRL1 (SEQ ID NO:4) is a PACAP27-specific receptor which mediates the up-regulation of CD11b and chemotactic migration, like other FPRL1 (SEQ ID NO:4) agonists, e.g., WKYMVm (SEQ ID NO:5), LL-37, and LXA4. Furthermore, FPRL1 (SEQ ID NO:4) mediates the PACAP27-induced calcium signaling in human monocytes (FIG. 2H) and U937 monocytic cell lines. Taken together, it may be suggested that FPRL1 (SEQ ID NO:4) mediates the inflammatory activity of PACAP27 (SEQ ID NO:1), a finding that should help elucidate the complicated interactions of PACAP (SEQ ID NO:20) and immune cells.

Therefore, the inflammatory conditions may be improved or prevented by any means which can inactivate PACAP27 (SEQ ID NO:1) or inhibit PACAP27 (SEQ ID NO:1) from binding to FPRL1 (SEQ ID NO:4). PACAP27 (SEQ ID NO:1) or the activity of PACAP27 (SEQ ID NO:1) to binding to FPRL1 (SEQ ID NO:4) may be inhibited any PACAP27 (SEQ ID NO:1) antagonists. In an embodiment of the present invention, it is proved that about 100 nM or more of peptide WRWWWW (SEQ ID NO:6) completely can inhibit the PACAP27-induced neutrophil activation, since the peptide binds to FPRL1 (SEQ ID NO:4) competitive with PACAP27 (SEQ ID NO:1). Further, in other embodiment of the present invention, it can be shown that amino acids “AA” positioned on 24^{th} and 25^{th} positions of C-terminus of PACAP27 (SEQ ID NO:1) plays an important role on binding to FPRL1 (SEQ ID NO:4). Therefore, a modification (e.g., deletion, or substitution or insertion with other amino acids) of the 24th and 25th amino acids of PACAP27 (SEQ ID NO:1), or a binding of other molecule to the amino acids may result in inactivating the activity of PACAP27 (SEQ ID NO:1) to bind to FPRL1 (SEQ ID NO:4). Further, GPCR inhibitors [e.g., pertussis toxin (PTX)], or a phospholipase C (PLC) inhibitors (e.g., U73122) also may inactivate the activity of PACAP27 (SEQ ID NO:1) through blocking a PACAP27-mediated calcium signaling.

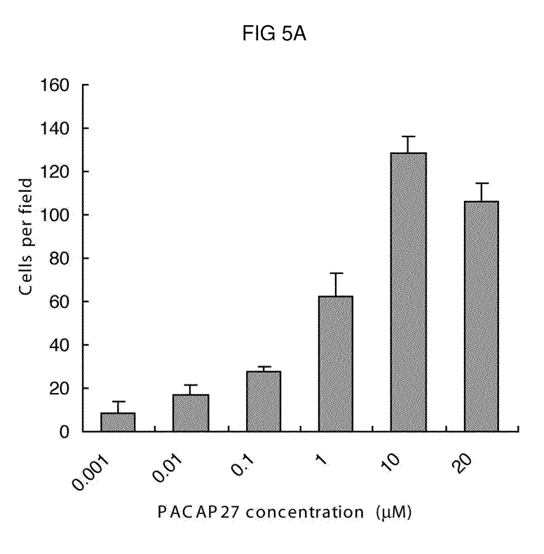

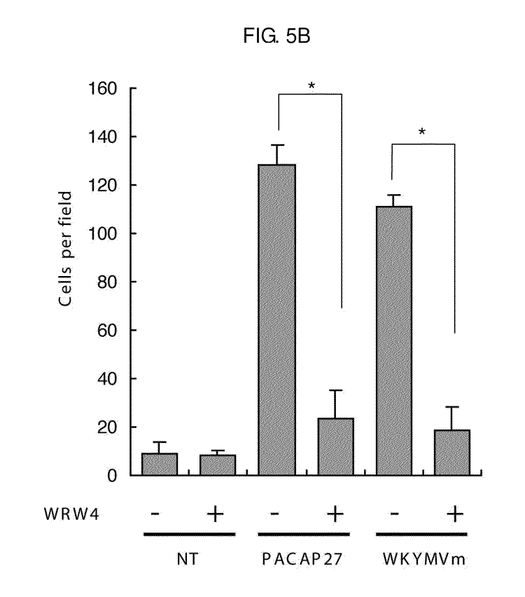

Previously, Cardell and colleagues demonstrated that PACAP38 (SEQ ID NO:2) or VIP (SEQ ID NO:3) inhibit fMLP-induced neutrophil chemotaxis (Kinhult, J., R. Uddman, M. Laan, A. Linden, and L. O. Cardell. 2001. Peptides. 22:2151-2154). Because in the present invention, it is shown that PACAP27 (SEQ ID NO:1) induced neutrophil chemotaxis and FPRL1 (SEQ ID NO:4) are required for this process (see FIG. 5), it is interesting to recall that the two different PACAPs have different effects on neutrophil chemotaxis. Although the inhibitory effects of PACAP38 (SEQ ID N0:2) and VIP (SEQ ID NO:3) on chemotaxis are not certainly elucidated, it can be speculated that VPAC1 might mediate an inhibitory effect, since it is expressed in neutrophils (Harfi, I., S. D'Hondt, F. Corazza, and E. Sariban. 2004. J. Immunol. 173: 4154-4163). It would be interesting to know the physiological relevance for the opposing role of PACAP38 (SEQ ID NO:2) and PACAP27 (SEQ ID NO:1) on the regulation of neutrophil chemotaxis.

Although no report has mentioned the pathophysiological relevance of the relation between PACAP molecules and neutrophils, some evidence is available in the literature. In particular, in the nasal cavity, PACAP molecules are known to affect glandular secretion (Hegg, C. C., E. Au, A. J. Roskams, and M. T. Lucero. 2003. J. Neurophysiol. 90:2711-2719). Interestingly, neutrophils are found in nasal cavity, and have been reported to play a major role in inflammatory disease in the nasal cavity (Nagakura, T., T. Onda, Y. Iikura, T. Masaki, H. Nagakura, and T. Endo. 1989. Allergy Proc. 10: 233-235). Therefore, it is possible that the local concentration of PACAP is markedly elevated in the nasal cavity under some conditions. However, no report is available on PACAP level changes with respect to the pathologic condition of the nasal cavity, and studies on disease-related PACAP27 (SEQ ID NO:1) changes are required to reveal the physiological role of PACAP27 (SEQ ID NO:1) with respect to the control of neutrophil behavior.

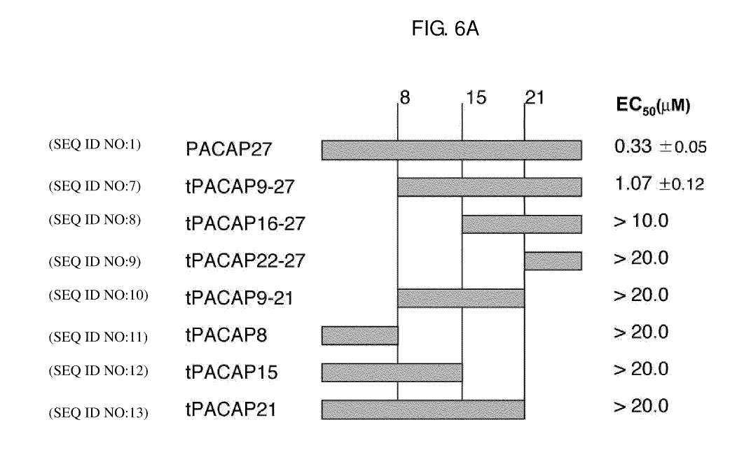

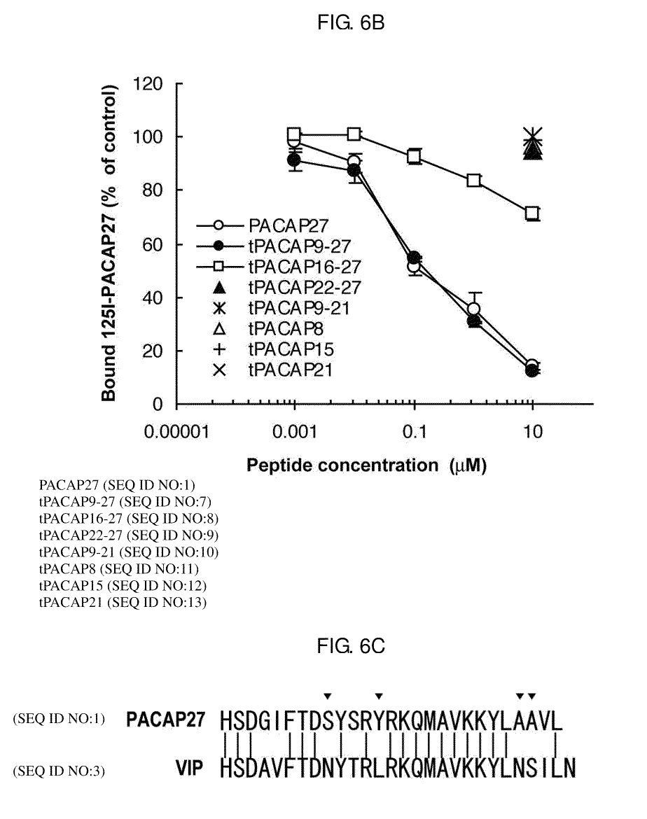

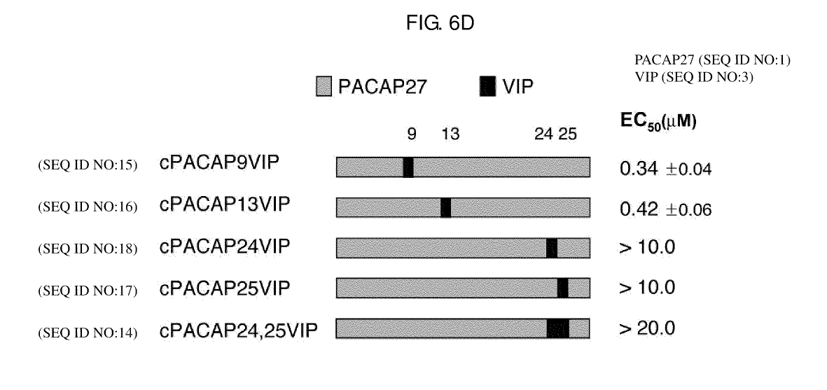

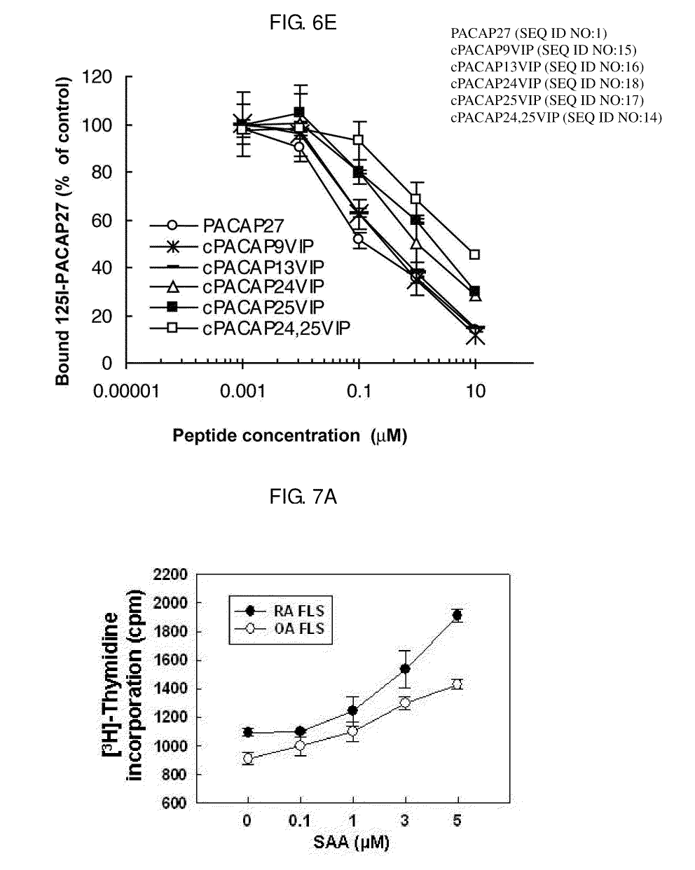

Recently structurally important motifs were identified to participate in the interaction between PAC1 and PACAP (SEQ ID NO:20). Specifically, the N-terminal region of PACAP is critical for receptor activation, and the C-terminal region for binding affinity (Inooka, H., T. Ohtaki, O. Kitahara, T. Ikegami, S. Endo, C. Kitada, K. Ogi, H. Onda, M. Fujino, and M. Shirakawa. 2001. Nat. Struct. Biol. 8:161-165). Therefore, PAC1 shows a similar affinities and sensitivities to PACAP27 (SEQ ID N0:1) and PACAP38 (SEQ ID NO:2). In the present invention, the interaction between FPRL1 (SEQ ID NO:4) and PACAP27 (SEQ ID NO:1) is demonstrated through the use of truncated or chimeric PACAP analogues (see FIG. 6). The C-terminal region of PACAP27 (SEQ ID NO:1) is crucial for both binding and activation, and the 11 additional residues in PACAP38 (SEQ ID NO:2) might hinder its binding to FPRL1 (SEQ ID NO:4) and thus facilitate PACAP27 (SEQ ID NO:1) selectivity. Although in the present invention, PACAP27 selective behavior in immune cells is revealed for the first time, similar activity has been reported in rat smooth muscle cells (Cox, H. M. 1992. Br. J. Pharmacol. 106:498-502; Ekblad, E., and F. Sundler. 1997. Eur. J. Pharmacol. 334:61-66), and though FPRL1 (SEQ ID NO:4) has not been described in smooth muscle cells, one group reported that fMLP, an agonist of FPR and FPRL1 (SEQ ID NO:4), induces transient coronary arterial muscle contraction (Keitoku, M., M. Kohzuki, H. Katoh, M. Funakoshi, S. Suzuki, M. Takeuchi, A. Karibe, S. Horiguchi, J. Watanabe, S. Satoh, M. Nose, K. Abe, H. Okayama, and K. Shirato. 1997. J. Mol. Cell. Cardiol. 29:881-894). Therefore, the present invention is important in that the direct relation between FPRL1 (SEQ ID NO:4) and PACAP27-selective response in smooth muscle cell should be understood.

GPCRs are classified into subfamilies according to their amino acid and nucleotide sequences. In general, GPCR subfamilies have similar ligands and binding motifs. For example, although sphingosine-1-phosphate is able to activate several receptors, these belong to the same rhodopsin-like GPCR subfamily. Opioid receptors, also members of the rhodopsin-like GPCRs family, are activated by multiple opioid peptides and share binding motif sequences. Interestingly, FPRL1 (SEQ ID NO:4) and the original PACAP receptors, PAC1, VPAC1, and VPAC2, belong to different subfamilies. That is, PAC1, VPAC1, and VPAC2 are members of the secretin-like GPCR subfamily, whereas FPRL1 (SEQ ID NO:4) is a rhodopsin-like GPCR. Furthermore, FPRL1 (SEQ ID NO:4) and PAC1 use different motifs to bind PACAP27 (SEQ ID NO:1). Taken together, it may be suggested that PACAP27-FPRL1 coupling presents a novel model of GPCR-ligand interaction.

In the present invention, it can be demonstrated that FPRL1 (SEQ ID NO:4) is a PACAP27-specific receptor, and it may be suggested that PACAP27 (SEQ ID NO:1) activates phagocytes via FPRL1 (SEQ ID NO:4) activation.

In another aspect, the present invention is to provide a complex of SAA-FPRL1 having a regulatory effect on immune response. Further, the present invention is to provide a composition of treating or preventing inflammatory diseases including Rheumatoid arthritis (RA), containing an inhibitor to inactivate the activity of SAA (SEQ ID NO:19) and/or FPRL1 (SEQ ID NO:4), or inhibitor of the binding of SAA (SEQ ID NO:19) to FPRL1 (SEQ ID NO:4) to inhibit the formation of the SAA-FPRL1 complex, wherein the composition has an inhibitory effect of synoviocyte hyperplasia and angiogenesis. Alternatively, the present invention is to provide a method of inhibiting synoviocyte hyperplasia and angiogenesis by inactivating the activity of SAA (SEQ ID NO:19) and/or FPRL1 (SEQ ID NO:4), or inhibiting the binding of SAA (SEQ ID NO:19) to FPRL1 (SEQ ID NO:4) to inhibit the formation of the SAA-FPRL1 complex, and a method of treating or preventing inflammatory diseases including RA by inactivating the activity of SAA (SEQ ID NO:19) and/or FPRL1 (SEQ ID NO:4), or inhibiting the binding of SAA (SEQ ID NO:19) to FPRL1 (SEQ ID NO:4) to inhibit the formation of the SAA-FPRL1 complex. Furthermore, the present invention is to provide a target for developing drugs treating or preventing inflammatory diseases including RA containing complex of SAA (SEQ ID NO:19) and FPRL1 (SEQ ID NO:4). The diseases include, but not limited to, atherosclerosis, Alzheimer's disease, cancer, and RA.

In the present invention, human SAA (NCBI accession no. P02735; SEQ ID NO:19) and human FPRL1 (NCBI accession no. P25090; SEQ ID NO:4) are employed. However, the amino acid sequences of SAA (SEQ ID NO:19) and FPRL1 (SEQ ID NO:4) are well conserved between different species, and thus, the present invention may be applied to all animals including the human being.

Serum amyloid A (SAA; SEQ ID NO:19) is a major acute-phase reactant. The present invention investigates the role of SAA (SEQ ID NO:19) in synovial hyperplasia and proliferation of endothelial cells, a hallmark pathological characteristic of rheumatoid arthritis (RA). In the present invention, it is revealed that SAA (SEQ ID NO:19) promotes the proliferation of fibroblast-like synoviocytes (FLS). In addition, SAA (SEQ ID NO:19) protects RA FLS against the apoptotic death induced by serum starvation, anti-Fas IgM, and sodium nitroprusside. The activity of SAA appears to be mediated by the formyl peptide receptor-like 1 (FPRL1; SEQ ID NO:4) receptor, as it was mimicked by the agonist peptide of FPRL1 (SEQ ID NO:4), but completely abrogated via the down-regulation of the FPRL1 (SEQ ID NO:4) transcripts by short interfering (si) RNA. The effect of SAA (SEQ ID NO:19) on FLS hyperplasia is shown to be mediated by an increase in the levels of intracellular calcium, as well as the activation of ERK and Akt, which resulted in an elevation in the expression of cyclin D1 and Bcl-2. Moreover, SAA (SEQ ID NO:19) stimulates the proliferation, migration, and tube formation of endothelial cells in vitro, and enhanced the sprouting activity of endothelial cells in both ex vivo and in vivo neovascularization. These observations indicate that the binding of SAA (SEQ ID NO:19) to FPRL1 (SEQ ID NO:4) may contribute to the destruction of bone and cartilage via the promotion of synoviocyte hyperplasia and angiogenesis, thus providing a potential target for the control of RA

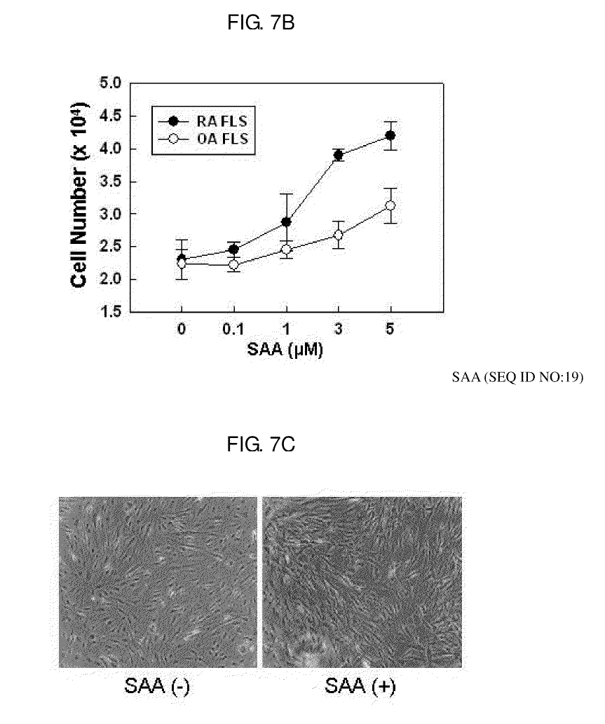

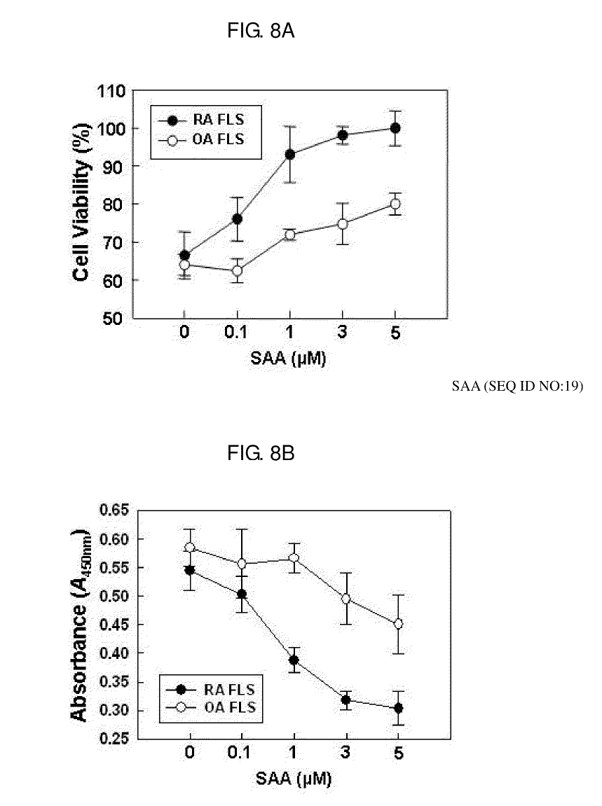

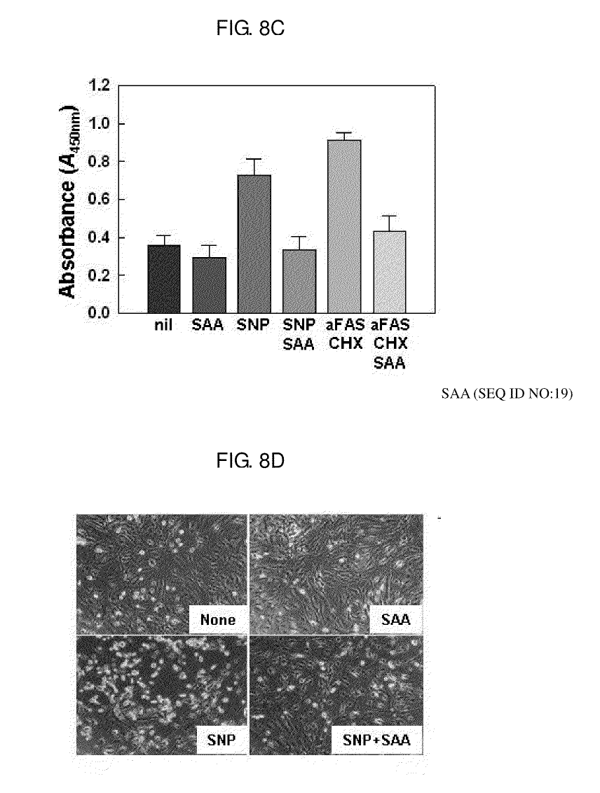

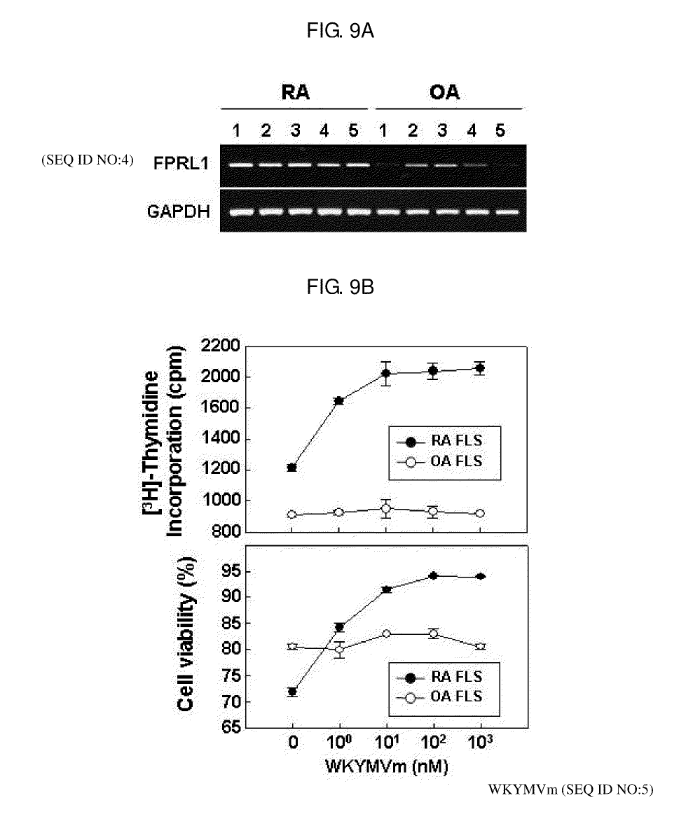

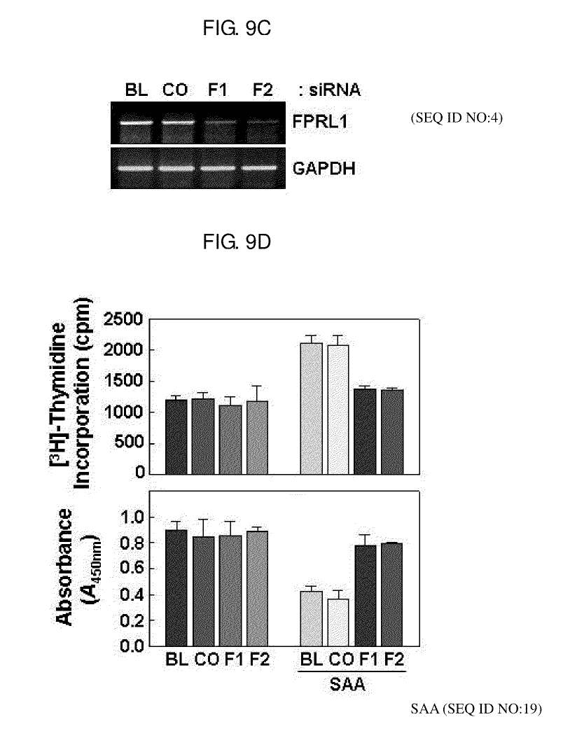

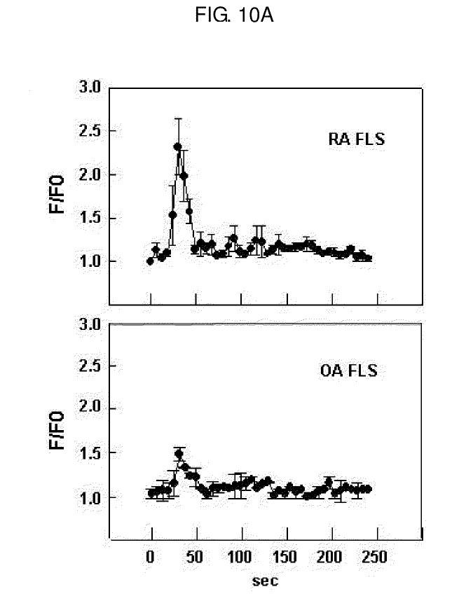

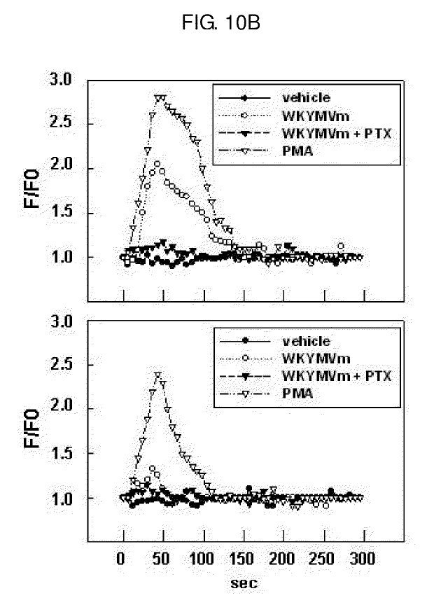

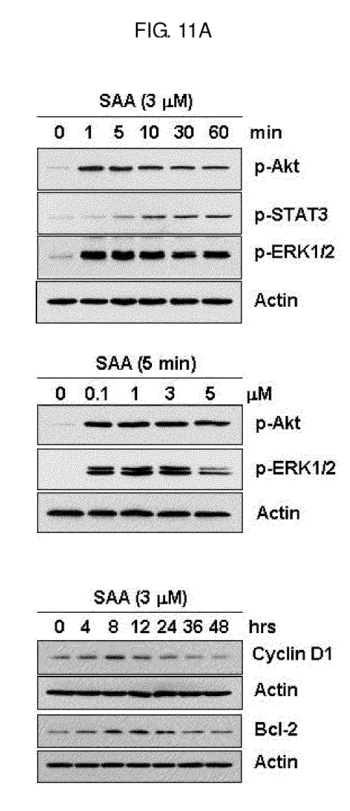

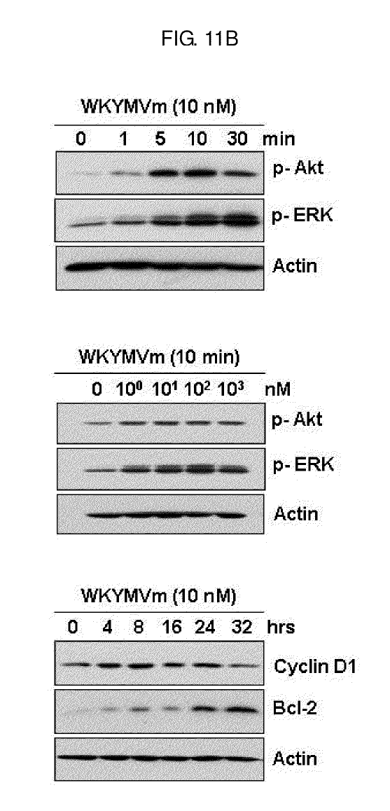

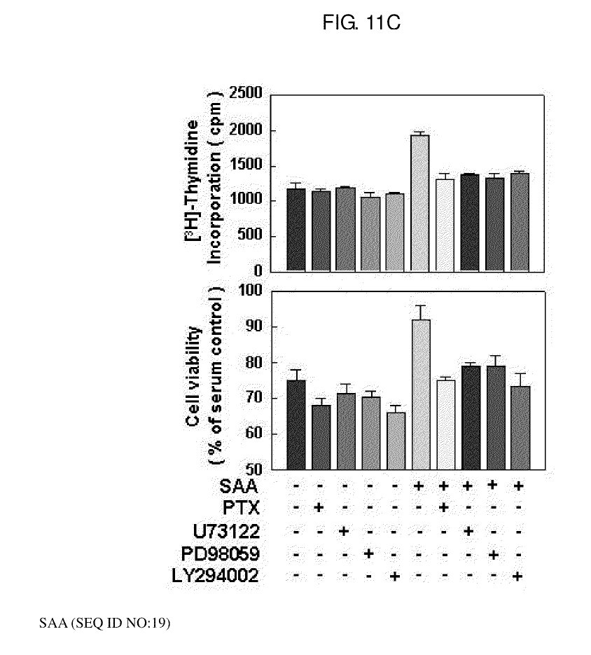

However, very little data is currently available regarding the functions of SAA (SEQ ID NO:19) in cellular proliferation and survival, as well as its intracellular targets. Therefore, in the present invention, it is shown that SAA (SEQ ID NO:19) stimulates the proliferation of FLS (see FIGS. 7A-7C). SAA (SEQ ID NO:19) has also been shown to prevent RA FLS against the apoptotic death induced by serum starvation, SNP, or anti-Fas IgM (see FIGS. 8A-8D). SAA-induced increases in the proliferation and survival of FLS is mimicked by the FPRL1 (SEQ ID NO:4) specific ligand, WKYMVm (SEQ ID NO:5) (see FIGS. 10A-10B). The activity of SAA (SEQ ID NO:19) on the proliferation and survival of FLS appears to be mediated by FPRL1 (SEQ ID NO:4), as it is abrogated completely by specific blockades of FPRL1 (SEQ ID NO:4) induced via treatment with siRNA (see FIGS. 9A-9D). SAA (SEQ ID NO:19) also increases the expression of cyclin D1 and Bcl-2 in rheumatoid synoviocytes (see FIGS. 11A-11C), which are critical for cell proliferation and survival, respectively, as well as the levels of p-ERK (phosphorylated ERK) and p-Akt (phosphorylated Akt), both of which are located upstream of the cyclin D1 and Bcl-2 signaling pathways. Moreover, the proliferative and anti-apoptotic activities of SAA (SEQ ID NO:19) are blocked completely via treatment with pharmacological ERK and Akt inhibitors (see FIGS. 11A-11C). Collectively, these data indicate that the interaction between SAA (SEQ ID NO:19) and FPRL1 (SEQ ID NO:4) induces the proliferation and survival of rheumatoid synoviocytes, via the ERK and Akt pathways.

Such results also indicated that the ability of SAA (SEQ ID NO:19) to promote both cell proliferation and survival was higher in the RA FLS than in the OA (osteoarthritis) FLS (see FIGS. 7A-7C and FIG. 8A-8D), thereby suggesting that RA FLS is more susceptible to SAA (SEQ ID NO:19) stimulation. This hyper-responsiveness to SAA (SEQ ID NO:19) may be attributable to the increased expression of FPRL1 (SEQ ID NO:4) in the RA FLS, as compared to the OA FLS, as observed in the present invention (see FIG. 9A). Several pro-inflammatory cytokines, including TNF-α, IL-β and IL-6, upregulate FPRL1 (SEQ ID NO:4) and SAA (SEQ ID NO:19) expression in RA FLS. Therefore, these cytokines may indirectly affect SAA (SEQ ID NO:19) response via the in vivo upregulation of FPRL1 (SEQ ID NO:4). Another possible explanation may involve differences in the SAA-evoked signal transduction pathway between the RA FLS and OA FLS (see FIGS. 8A and 8B). These increases in [Ca²⁺]ᵢ levels, as well as the activation of ERK and Akt, may more potently stimulate the expression of cyclin D1 and Bcl-2, resulting in enhanced proliferation and survival. Due to the elevated SAA (SEQ ID NO:19) and FPRL1 (SEQ ID NO:4) expression levels in RA-afflicted joints as compared to OA joints, the Ca²⁺ response and the activation of signaling molecules, most notably ERK and Akt, might be accentuated or further prolonged under in vivo arthritic conditions.

The supply of sufficient oxygen and nutrients via neovascularization is required for the perpetuation of synovial hyperplasia. Furthermore, the newly-formed blood vessels provide a surface to which leukocytes can adhere and through which they can migrate, delivering more inflammatory cells and molecules to arthritic lesions. Therefore, angiogenesis is essential to the progression of chronic arthritis, and also constitutes an early determinant of RA.

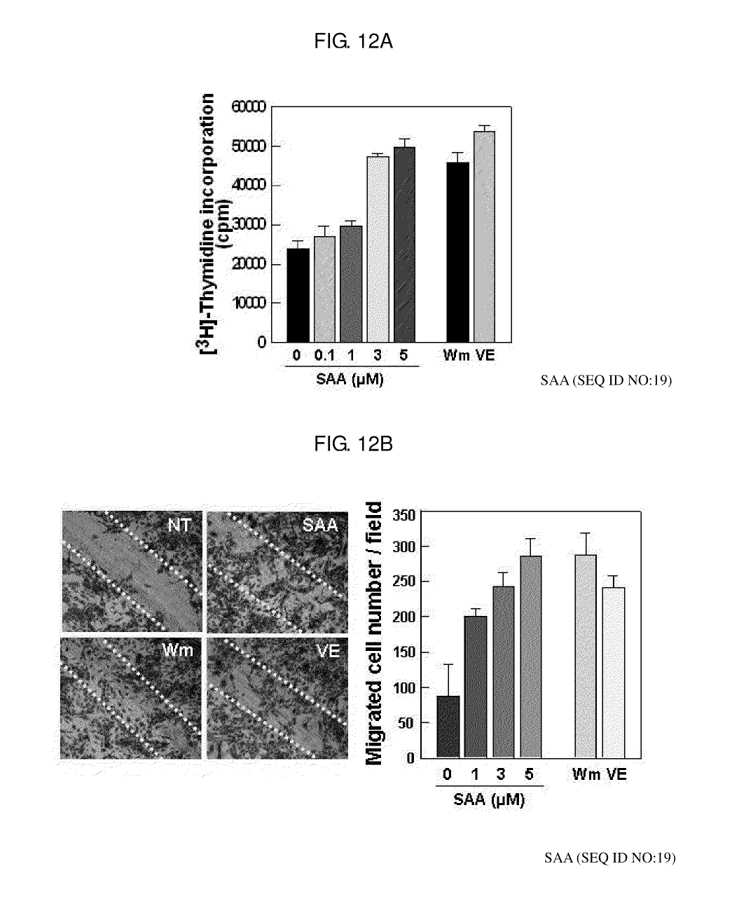

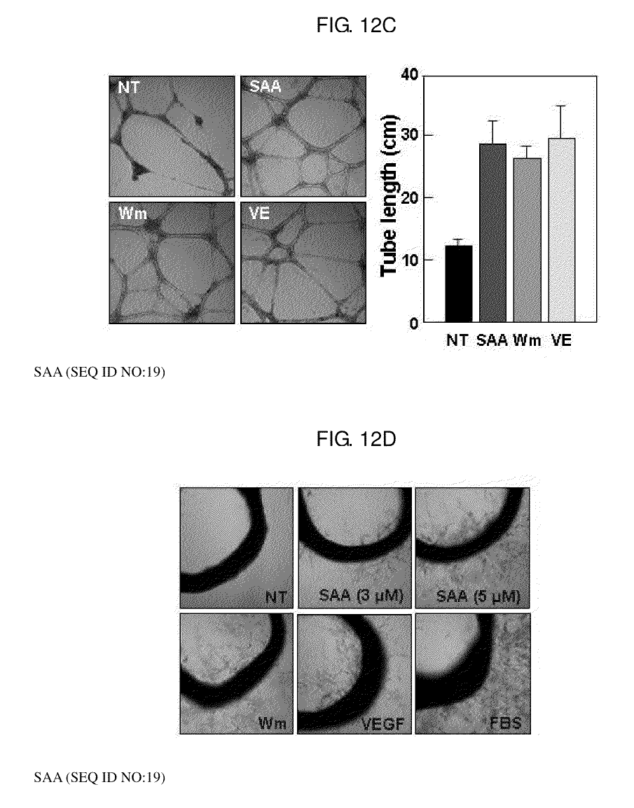

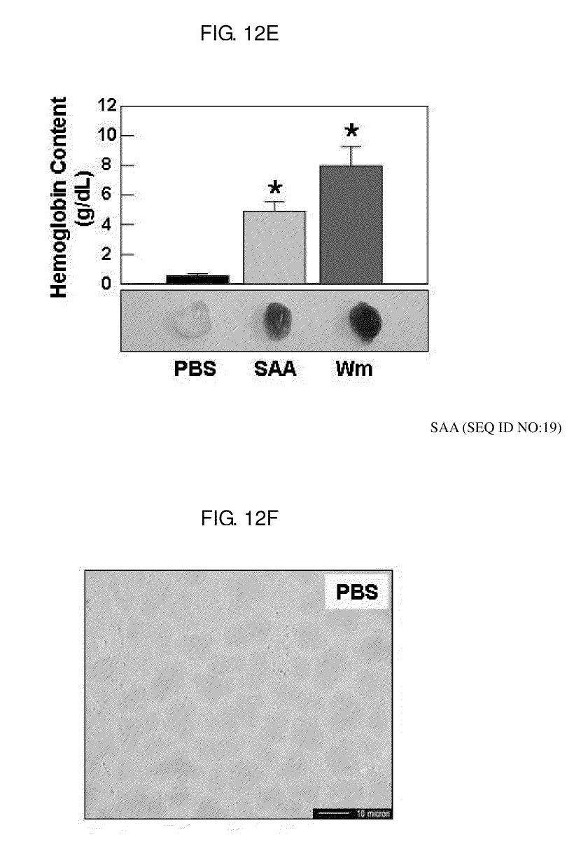

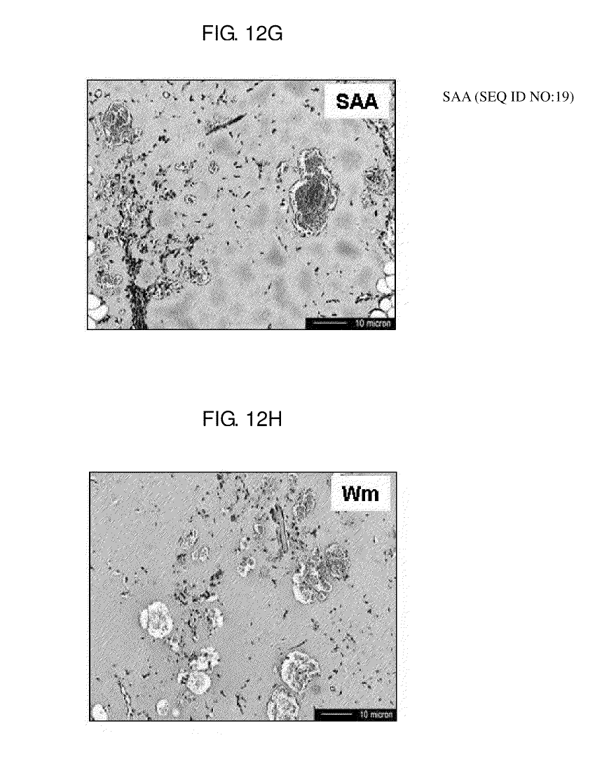

The functions of SAA (SEQ ID NO:19) in endothelial proliferation, as well as its in vivo effects on angiogenesis, remain to be clearly elucidated. In the present invention, it is determined that SAA (SEQ ID NO:19) stimulated proliferation, migration, and the formation of capillary tubes in vitro (see FIGS. 12A-12H). Moreover, the sprouting of endothelial cells is found to be up-regulated by SAA (SEQ ID NO:19) treatment in an ex vivo rat aorta sprouting assay (see FIGS. 12A-12H). The angiogenic activity of SAA (SEQ ID NO:19) is confirmed by the results of an in vivo mouse Matrigel plug assay (see FIGS. 12A-12H). Collectively, the findings of the present invention, coupled with the findings of an earlier report, suggest that, in RA patients, SAA (SEQ ID NO:19) may facilitate the destruction of joints via the promotion of angiogenesis.

There are several potential mechanisms whereby SAA (SEQ ID NO:19) might exert positive effects on the survival characteristics of synoviocytes. First, as suggested above, SAA (SEQ ID NO:19), which is generated primarily by macrophages, endothelial cells, and synoviocytes, can exert an inhibitory effect on the apoptotic death of FLS, while inducing heightened cellular proliferation. Second, SAA (SEQ ID NO:19) may participate indirectly in the survival characteristics of synoviocytes, via the activation of inflammatory cascades. For example, SAA (SEQ ID NO:19) may recruit leukocytes in the synovial membrane, in which newly-employed leukocytes might induce the proliferation of synoviocytes via cell-to-cell contact. Thirdly, SAA (SEQ ID NO:19) promotes angiogenesis, which may diminish the growing burden of the synoviocytes, via the supply of oxygen and nutrients for tissue metabolism. As a result, expanded FLS may secrete elevated quantities of SAA (SEQ ID NO:19), which would then further stimulate the proliferation of FLS in an autocrine or paracrine manner, thereby constructing a positive feedback loop. Taking this into account, SAA (SEQ ID NO:19) may be considered to be a critical mediator of pannus formation, and thus the development of an antagonist that would block the activity of SAA (SEQ ID NO:19) or FPRL1 (SEQ ID NO:4), might eventually prove useful with regard to the development of a treatment for RA. Such a possibility is currently under study and consideration.

In conclusion, in the present invention, SAA (SEQ ID NO:19) is shown to induce the proliferation of both FLS and endothelial cells, via its binding to its receptor, FPRL1 (SEQ ID NO:4). SAA (SEQ ID NO:19) is also shown to exert a protective effect against synoviocyte apoptosis in RA-afflicted joints. The cytoprotective and proliferative activity of SAA is achieved via the stimulation of intracellular Ca²⁺, ERK and Akt activity in the FLS.

Therefore, synoviocyte hyperplasia and angiogenesis may be effectively inhibited by blocking the binding of SAA (SEQ ID NO:19) to FPRL1 (SEQ ID NO:4), activation of SAA (SEQ ID NO:19), or intracellular Ca²⁺, ERK or Akt activity, whereby inflammatory diseases induced by synoviocyte hyperplasia and/or angiogenesis can be treated or prevented. For example, the binding of SAA (SEQ ID NO:19) to FPRL1 (SEQ ID NO:4) and activation of SAA (SEQ ID NO:19) may be inhibited by, but not limited to, one or more inhibitors selected from the group consisting of SAA antagonists, anti-FPRL1 antibodies for blocking of SAA binding to FPRL1, GPCR inhibitors (e.g., PTX), ERK inhibitors (e.g., PD98059), or AKT inhibitors (e.g., LY294002) for blocking of the activation of intracellular signaling by SAA (SEQ ID NO:19), respectively.

The findings of the present invention suggest that the interaction occurring between SAA (SEQ ID NO:19) and FPRL1 (SEQ ID NO:4) may be critical with regard to the hyperplasia of rheumatoid synoviocytes, and may also have important implications in terms of abnormal synoviocyte growth and therapeutic intervention in cases of RA.

The present invention is further explained in more detail with reference to the following examples. These examples, however, should not be interpreted as limiting the scope of the present invention in any manner.

### Example 1

### Investigation of PACAP27 Activities

1.1. Materials

PACAP27 (SEQ ID NO:1), PACAP38 (SEQ ID NO:2), and VIP (SEQ ID NO:3) were obtained from Phoenix Pharmaceuticals, Inc. (Belmont, Calif.). Truncated PACAPs were synthesized by the Peptide Library Support Facility (Pohang, Korea). Chimeric PACAPs were purchased from GenScript (Piscataway, N.J.); radioiodinated PACAP27 (SEQ ID NO:1) (125I-labeled) from Perkin-Elmer (Boston, Mass.); and peripheral blood mononuclear cell separation medium (Histopaque-1077) from Sigma (St. Louis, Mo.). RPMI1640 medium and high glucose Dulbecco's modified Eagle's medium (DMEM) were obtained from Invitrogen (Carlsbad, Calif.); dialyzed fetal bovine serum from Hyclone Laboratories (Logan, Utah); fura-2 pentaacetoxymethylester (fura-2/AM) from Molecular Probes (Eugene, Oreg.); anti-phospho-ERK antibodies and anti-ERK2 antibodies from Cell Signaling (Beverly, Mass.); phcoerythrine (PE)-labeled human CD11b-antibodies from BD PharMingen (San Diego, Calif.); Limulus Amebocyte Lysates assay (QCL-1000) from Cambrex Bio Science (Walkersville, Md.); polymyxin b from Sigma (St. Louis, Mo.); and chemotaxis multiwell chambers from Neuroprobe (Gaithersburg, Md.).

1.2. Cell Culture

FPRL1-expressing rat basophile leukemia (RBL)-2H3 (FPRL1/RBL), FPR-expressing RBL-2H3 (FPR/RBL), and vector-transfected RBL-2H3 (vector/RBL) were donated by Dr. Richard D. Ye (University of Illinois). FPRL1/RBL, FPR/RBL, and vector/RBL were maintained at 37° C. in a humidified 5% CO₂ atmosphere in high glucose DMEM supplemented with 20% (vol/vol) heat-inactivated fetal calf serum and G418 (500 g/mL). FPRL1/RBL, FPR/RBL, and vector/RBL were sub-cultured every three days. The prepared cells were used in the following examples.

1.3. Preparation of Neutrophils and Monocytes Peripheral blood was collected from healthy donors (male, 20˜30 years old, venous blood collection). Human neutrophils were isolated by dextran sedimentation followed by hypotonic erythrocyte lysis and lymphocyte separation medium gradient, as described in “Bae, Y. S., H. Bae, Y. Kim, T. G. Lee, P. G. Suh, and S. H. Ryu. 2001. Identification of novel chemoattractant peptides for human leukocytes. Blood 97:2854-2862”. Isolated human neutrophils were used promptly. Peripheral blood mononuclear cells (PBMCs) were separated on a Histopaque-1077 gradient (Bae, Y. S., H. Bae, Y. Kim, T. G. Lee, P. G. Suh, and S. H. Ryu. 2001. Identification of novel chemoattractant peptides for human leukocytes. Blood 97:2854-2862). After twice washing with Hanks' balanced salt solution (HBSS, Invitrogen, Carlsbad, Calif.) without Ca²⁺ and Mg²⁺, the PBMCs were then suspended in 10% FBS containing RPMI 1640 medium (Invitrogen, Carlsbad, Calif.) and incubated for 60 min at 37° C. to let the monocytes attach to the culture dish. The cells were washed five times with warmed RPMI 1640 medium to wash out lymphocytes and then the attached monocytes were collected as described in above Bae, Y. S. et al.

1.4. PACAP27 Activity of Specifically Stimulating Intracellular Signaling in Human Neutrophils

The expressions of PACAP receptors in immune cells have been reported by several groups, but their functions are unclear. Here, the present example is to find the functions by measuring intracellular calcium concentration, intracellular cyclic AMP and ERK phosphorylation, as below.

Neutrophils were stimulated with 1 μM of PACAP27 (SEQ ID NO:1), PACAP38 (SEQ ID NO:2), or VIP (SEQ ID NO:3), and calcium concentrations were measured for 10 min. In order to measure EKR phosphorylation, each peptide hormones was used with 1 μM concentrations for 5 min. For cAMP measurement, each peptide hormones was used with 1 μM concentrations for 10 min.

1.4.1. Intracellular Calcium Mobilization Measurements

Intracellular calcium concentrations ([Ca²⁺]ᵢ) were determined using Grynkiewicz's method with fura-2/AM, as described in “Grynkiewicz, G., M. Poenie, and R. Y. Tsien. 1985. A new generation of Ca²⁺ indicators with greatly improved fluorescence properties. J. Biol. Chem. 260:3440-3450”. Briefly, the cells prepared in Example 1.2 were incubated with 3 M fura-2/AM at 37° C. for 50 minutes in fresh serum free RPMI 1640 medium with continuous stirring. The incubated cells (2×10⁶) were aliquoted for each assay in Ca²⁺-free Locke's solution (154 mM NaCl, 5.6 mM KCl, 1.2 mM MgCl₂, 5 mM HEPES (pH 7.3), 10 mM glucose, and 0.2 mM EGTA). Fluorescence changes at 340 and 380 nm using a common emission wavelength of 500 nm were measured, and fluorescence ratios were converted to [Ca2+]ᵢ, as described in above Grynkiewicz, G. et al.

1.4.2. Intracellular Cyclic AMP Measurements

Briefly, neutrophils were isolated and resuspended at 5×10⁶ cells/ml in Hank's balanced salt solution (HBSS) for 5-10 minutes in a shaking incubator. The HBSS was then replaced with 100 ml HBSS containing 500 M isobutylmethylxanthine (IBMX; a cAMP phosphodiesterase inhibitor) for 5 minutes, and then cells were stimulated for 10 minutes. The reaction was terminated by adding 1 ml of ethanol, and cAMP levels were determined by using cAMP measuring kit (Neurunex, Pohang, Korea) according to the manufacturer's instructions. From this result, it could be concluded that the PACAP27-induced immune cell activation is independent to cAMP signaling cascade.

1.4.3. Western Blot Analysis for ERK Phosphorylation

ERK phosphorylation levels were measured by Western blotting, as described in above Bae, Y. S. et al. Cells (2×10⁶/assay) were stimulated with the indicated concentration of agonist for 5 minutes, then washed with serum-free RPMI 1640 medium and lysed in lysis buffer {20 mM HEPES (pH 7.2), 10% glycerol, 150 mM NaCl, 1% Triton X-100, 1 mM PMSF, 10 g/ml leupeptin, 10 g/ml aprotinin, 50 mM NaF, and 1 mM Na3VO4}. Detergent-insoluble materials were pelleted by centrifugation (12,000×g, 15 minutes, 4° C.), and the soluble supernatant fraction was removed and either stored at −80° C. or used immediately. Laemmli sample buffer was added to these fractions and boiled (5 minutes). Proteins were separated by SDS-PAGE and transferred to nitrocellulose membranes (Schleicher and Schuell, BA85). Blocking was performed using TBS buffer (10 mM Tris/HCl, pH 7.5, 150 mM NaCl, and 0.05% Tween-20) containing 5% nonfat dry milk. Membranes were probed with a phospho-ERK specific primary antibody or ERK2 antibody for 3 hours at room temperature. Subsequently immunoblots were washed and incubated with a horseradish peroxidase-linked secondary antibody (Kirkegaad and Perry Laboratories, Gaithersburg, Md.) for 1 hour at room temperature, rinsed four times in TBS buffer, and then developed with horseradish peroxidase-dependent chemiluminescence reagents (Amersham International, United Kingdom). In this example, it can be shown that ERK is successfully phosphorylated by PACAP27 treatment.

1.4.4. Result

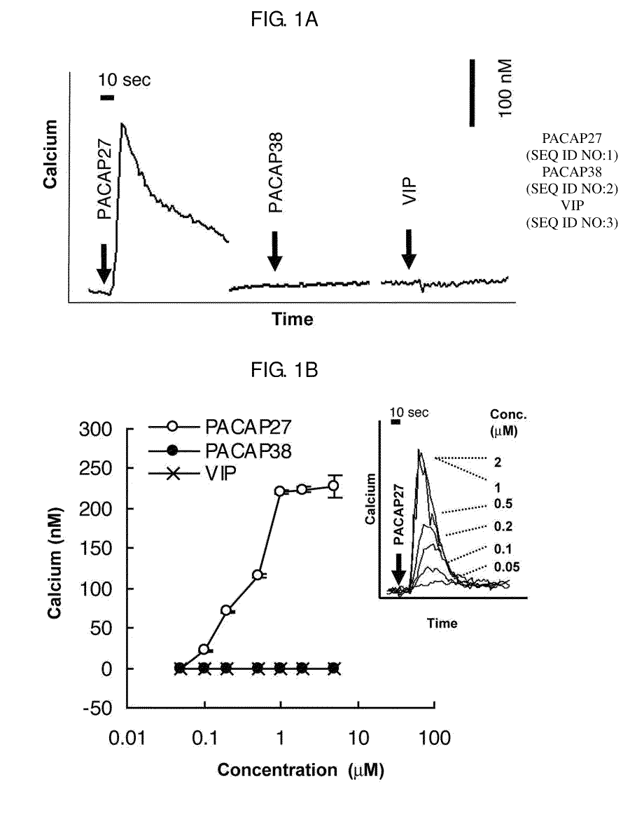

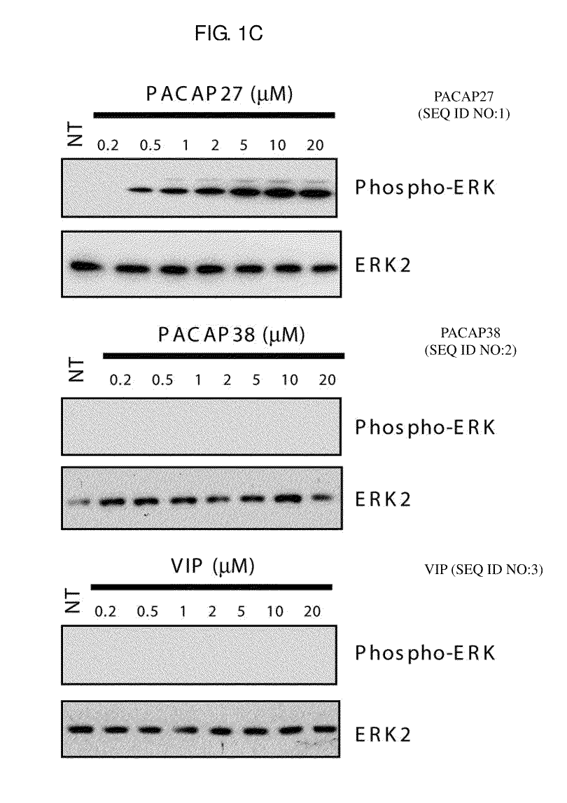

It was found that the stimulation of human neutrophils with 1 M PACAP27 (SEQ ID NO:1) profoundly increased [Ca2+]I, as shown in FIG. 1A. However, neither PACAP38 (SEQ ID NO:2) nor VIP (SEQ ID NO:3) increased [Ca2+]ᵢ. In order to confirm this PACAP27-specific activation, the dose dependencies of PACAP27 (SEQ ID NO:1), PACAP38 (SEQ ID NO:2), or VIP (SEQ ID NO:3) were examined, and it was found that only PACAP27 (SEQ ID NO:1) increased [Ca2+]ᵢ (see FIG. 1B). At 100 nM PACAP27 (SEQ ID NO:1) induced a significant [Ca2+]ᵢ increase (see FIG. 1B inset). PACAP27-induced signaling was also observed to be associated with the dose-dependent phosphorylation of ERK (FIG. 1C). These data suggest that PACAP27 (SEQ ID NO:1) specifically stimulates human neutrophils. In view of the fact that VPAC1 can be stimulated by VIP (SEQ ID NO:3) or PACAP38 (SEQ ID NO:2), as well as by PACAP27 (SEQ ID NO:1), these results were not consistent with those of a previous report, which suggested that VPAC1 functions as a PACAP receptor in neutrophils (Harfi, I., S. D'Hondt, F. Corazza, and E. Sariban. 2004. J. Immunol. 173:4154-4163). These data suggest that another receptor may be involved in the process of PACAP27-induced intracellular signaling in human neutrophils.

In Example 1, the results are expressed as means±SE. In the figure legends, * indicates p<0.01 versus the appropriate vehicle treated control.

1.5. FPRL1 (SEQ ID NO:4) as a Specific Receptor for PACAP27 (SEQ ID NO:1)

1.5.1. Ligand Binding Analysis

To find another receptor for PACAP27 (SEQ ID NO:1), a ligand binding analysis was performed as follows:

The ligand binding analysis was performed as described in “Bae, Y. S., H. Y. Lee, E. J. Jo, J. I. Kim, H. K. Kang, R. D. Ye, J. Y. Kwak, and S. H. Ryu. 2004. J. Immunol. 173:607-614”. Briefly, FPRL1/RBL cells were seeded at 1×10⁵ cells/well onto a 24 well plate and cultured overnight. After blocking them with blocking buffer (33 mM HEPES, pH 7.5, 0.1% BSA in RPMI 1640 medium) for 2 hours, 50 pM of ¹²⁵I-labeled PACAP27 (Perkin-Elmer, Boston, Mass.) was added to the cells in binding buffer (PBS containing 0.1% BSA), in the presence of the test peptides (cold PACAP27 (SEQ ID NO:1), truncated-PACAPs, and chimeric PACAPs), and then incubated for 3 hours at 4° C. with continuous shaking. The cells were then washed 5 times with ice-cold binding buffer, and 200 L of lysis buffer (20 mM Tris, pH 7.5, 1% Triton X-100) was added to each well for 20 minutes at room temperature. Lysates were then collected and counted using a γ-ray counter.

1.5.2. Result

To determine the characteristic properties of the PACAP27-specific receptor in human neutrophils, the effects of pertussis toxin (PTX, Sigma Aldrich) or U73122 [a specific phospholipase C (PLC) inhibitor, Sigma Aldrich) on PACAP27 (SEQ ID NO:1)-mediated calcium signaling were assessed as shown in FIGS. 2A and 2B.

Several chemoattractant receptors have been reported to exert stimulatory effects on neutrophils via PTX-sensitive GPCRs and by the activation of PLC (Bae, Y. S., H. Bae, Y. Kim, T. G. Lee, P. G. Suh, and S. H. Ryu. 2001. Blood 97:2854-2862). In order to determine whether PACAP27 (SEQ ID NO:1) can stimulate known chemoattractant receptors in human neutrophils, calcium signaling in response to sequential stimulation using PACAP27 (SEQ ID NO:1) and the known chemoattractants, fMLP, WKYMVm (SEQ ID NO:5), or C5a (see FIG. 2C) was analyzed. Treatment with 1 M PACAP27 (SEQ ID NO:1) and 10 nM WKYMVm (SEQ ID NO:5) resulted in bidirectional desensitization, suggesting that both ligands share the same receptor (FIG. 2C). Since WKYMVm (SEQ ID NO:5) stimulates members of the formyl peptide receptor (FPR) family, particularly FPRL1 (SEQ ID NO:4) at low nanomolar concentrations (17), the effects of PACAP27 (SEQ ID NO:1) on calcium signaling in RBL-2H3 cells expressing either FPR or FPRL1 (SEQ ID NO:4) (FPR/RBL or FPRL1/RBL) were examined. PACAP27 (SEQ ID NO:1) was found to exert a profound stimulatory effect on FPRL1/RBL cells, but not on vector/RBL or FPR/RBL cells (FIG. 2D). The effects of the FPRL1-selective antagonist, Trp-Arg-Trp-Trp-Trp-Trp (WRW4; SEQ ID NO:6) (15), on PACAP27-induced signaling in human neutrophils were also examined. WRW4 (SEQ ID NO:6) successfully inhibited PACAP27-induced [Ca2+]ᵢ up-regulation (FIG. 2E), but failed to inhibit PACAP27-induced cAMP elevation (FIG. 2G), indicating that WRW4 (SEQ ID NO:6) does not affect VPAC1, which has been reported to be expressed in human neutrophils (Harfi, I., S. D'Hondt, F. Corazza, and E. Sariban. 2004. J. Immunol. 173:4154-4163).

ERK-phosphorylation was also completely inhibited by pretreating with WRW4 (SEQ ID NO:6), indicating that this ERK phosphorylation is also a part of the FPRL1-dependent signaling cascade (FIG. 2F). Since monocytes were reported to express FPRL1 (SEQ ID NO:4) (Le, Y., W. Gong, B. Li, N. M. Dunlop, W. Shen, S. B. Su, R. D. Ye, and J. M. Wang. 1999. J. Immunol. 163:6777-6784), the effects of WRW4 (SEQ ID NO:6) on PACAP27-induced calcium signaling in human monocytes were also examined. WRW4 (SEQ ID NO:6) successfully inhibited PACAP27-induced calcium signaling in human monocytes (see FIG. 2H).

1.6. PACAP27 (SEQ ID NO:1) Primes fMLP-Induced Calcium Signaling in a FPRL1-Dependent Manner

PACAP27 (SEQ ID NO:1) has been reported to prime fMLP-induced calcium signaling (Harfi, I., S. D'Hondt, F. Corazza, and E. Sariban. 2004. J. Immunol. 173:4154-4163). To determine the FPRL1-dependency, the effect of PACAP27 (SEQ ID NO:1) on fMLP-induced calcium signaling with or without WRW4 (SEQ ID NO:6) were examined as follows. Changes at 340 nm and 380 nm were monitored and fluorescence ratios were converted to [Ca²⁺]ᵢ. Neutrophils were treated with vehicle or 1 M WRW4 (SEQ ID NO:6) for 30 seconds, prior to being stimulated with vehicle, 1 M PACAP27 (SEQ ID NO:1), 10 nM fMLP, or both. The results are shown in FIG. 3. The results shown are representative of two independent experiments performed in duplicate. *, p<0.01 vs control.

As shown in FIG. 3, fMLP-induced calcium signaling was not affected by WRW4 (SEQ ID NO:6), indicating that fMLP acts on FPR. PACAP27 (SEQ ID NO:1) notably enhanced fMLP-induced calcium signaling, and this event was abolished by WRW4 (SEQ ID NO:6) treatment, indicating the priming effect was FPRL1 (SEQ ID NO:4) dependent.

1.7. PACAP27 (SEQ ID NO:1) Induces CD11b Up-Regulation in Neutrophils in a FPRL1-Dependent Manner

It was examined whether PACAP27 (SEQ ID NO:1) stimulates the surface expression of CD11b, as follows.

1.7.1. FACS Analysis

Purified neutrophils were incubated with indicated concentration of PACAP27 (SEQ ID NO:1) for 1 hour. Cells (2×10⁵/assay) were washed with FACS buffer (PBS containing 1% BSA and 0.1% sodium azide), incubated with human AB type serum for 10 minutes on ice, and stained with PE-labeled human CD11b antibody (BD PharMingen, San Diego, Calif.). They were then analyzed using a FACSCalibur system (BD Biosciences, San Jose, Calif.), as described in “Harfi, I., S. D'Hondt, F. Corazza, and E. Sariban. 2004. J. Immunol. 173:4154-4163”.

1.7.2. Result

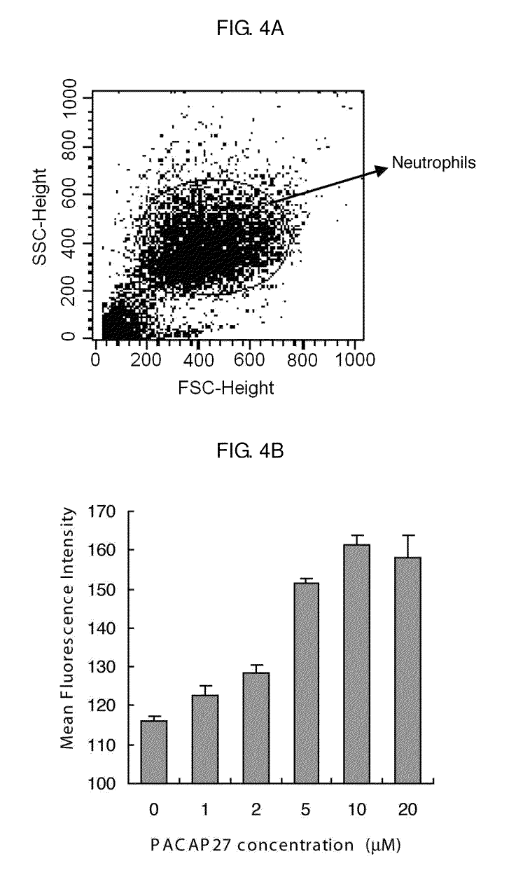

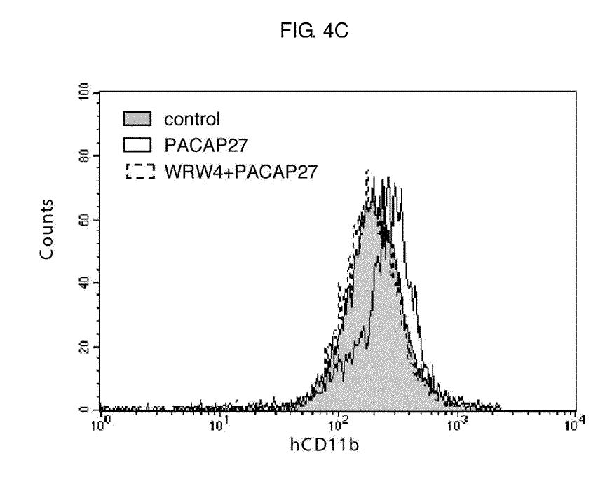

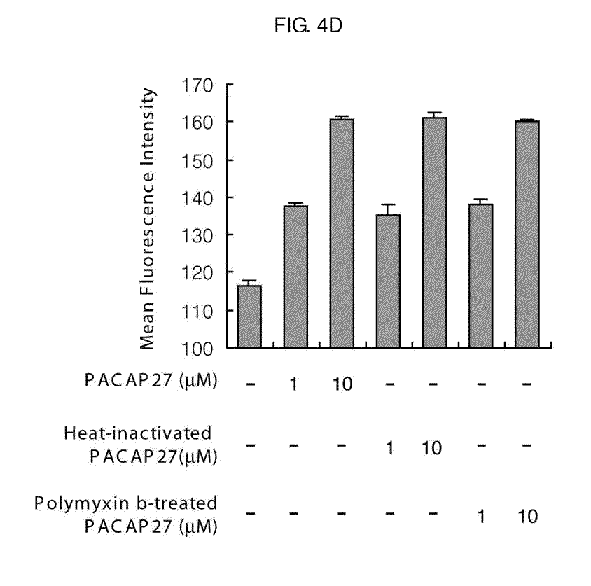

Purified neutrophils were incubated with PACAP27 (SEQ ID NO:1), and analyzed by flow cytometry, as shown by the dot plots in FIG. 4A. It was observed that PACAP27 (SEQ ID NO:1) up-regulated CD11b, maximally at 10 M (FIG. 4B). Moreover, CD11b up-regulation was inhibited completely by the FPRL1 antagonist, WRW4 (SEQ ID NO:6), indicating that it is a FPRL1-dependent process (FIG. 4C). In order to abolish the possibility of endotoxin contamination of PACAP27 (SEQ ID NO:1), We measured endotoxin content in PACAP27 (SEQ ID NO:1) sample via Limulus Amebocyte Lysates assay (QCL-1000, Cambrex Bio Science, Walkersville, Md.), and endotoxin was not detected (much less than 0.1 EU/mg, data not shown).

The heat-inactivation and polymyxin b (Sigma, St. Louis, Mo.) treatment on PACAP27 (SEQ ID NO:1) induced CD11b up-regulation were also tested. There is no difference among PACAP27 (SEQ ID NO:1), boiled PACAP27 (SEQ ID NO:1), and polymyxin b-treated PACAP27 (SEQ ID NO:1), indicating that the synthetic PACAP27 (SEQ ID NO:1) is endotoxin free

1.8. PACAP27 (SEQ ID NO:1) Induces the Chemotactic Migration of Neutrophils in a FPRL1-Dependent Manner

As FPRL1 (SEQ ID NO:4) participates in leukocyte migration in concert with several specific ligands, it was examined whether PACAP27 (SEQ ID NO:1) induces neutrophil chemotaxis by investigating the chemotactic migration of neutrophils.

1.8.1. Chemotaxis Assays

Chemotaxis assays were performed using multiwell chambers (Neuroprobe Inc., Gaithersburg, Md.) (Bae, Y. S., H. Bae, Y. Kim, T. G. Lee, P. G. Suh, and S. H. Ryu. 2001. Blood 97:2854-2862). Prepared human neutrophils were suspended in RPMI 1640 medium at a 1×10⁶ cells/ml, and 25 μl of this suspension was placed into the upper well of a chamber separated from the lower chamber, which was filled with testing solutions, by a 3 mm filter (not coated with polyvinylpyrrolidone). After incubating for 2 hours at 37° C., non-migrated cells were removed by scraping, and cells that had migrated across the filter were dehydrated, fixed, and stained with hematoxylin (Sigma, St. Louis, Mo.). Stained cells in five randomly chosen high power fields (HPF) (400×) were then counted.

1.8.2. Result

Neutrophil migration was analyzed for 2 hours across a polycarbonate membrane. Various concentrations of PACAP27 (SEQ ID NO:1) as shown in the following Table 1 were placed in the upper and lower compartments of the chambers. Data are presented as means±SE for migrated neutrophils cells per field counted in triplicate of two independent experiments.

TABLE 1
Checkboard analysis of neutrophil after
treatment with PACAP27 (SEQ ID NO: 1)

| | Above | | | |
|---|---|---|---|---|
| | | PACAP27 (M) | | |
| Below | Medium | 0.1 | 1 | 10 |
| Medium | 0 ± 0 | 0 ± 0 | 0 ± 0 | 0 ± 0 |
| PACAP27 (M) | | | | |
| 0.1 | 34.7 ± 2.5 | 19.3 ± 3.1 | 16.0 ± 5.7 | 16.9 ± 6.2 |
| 1 | 59.3 ± 1.0 | 33.7 ± 1.4 | 21.3 ± 3.4 | 20.7 ± 8.4 |
| 10 | 162.3 ± 31.0 | 147.7 ± 28.8 | 72.7 ± 11.5 | 73.7 ± 6.9 |

It was found that it elicited the chemotactic migration of neutrophils dose-dependently with maximal activity at 10 M as shown in FIG. 5A and Table 1. The involvement of FPRL1 (SEQ ID NO:4) in PACAP27-induced neutrophil chemotaxis was examined using the FPRL1 antagonist, WRW4 (SEQ ID NO:6). As shown in FIG. 5B, PACAP27-induced neutrophil chemotaxis was completely inhibited by WRW4 (SEQ ID NO:6), indicating that this process requires FPRL1 (SEQ ID NO:4) (FIG. 5B).

1.9. The C-terminal Region of PACAP27 is Important for its Interaction with FPRL1 (SEQ ID NO:4)

To characterize the interaction between PACAP27 (SEQ ID NO:1) and FPRL1 (SEQ ID NO:4), a number of truncated PACAPs (tPACAPs) were synthesized by deleting the N- or C-terminal sequences of PACAP27 (SEQ ID NO:1), as shown in FIG. 6A (SEQ ID NO:7. tPACAP9-27; SEQ ID NO:8, tPACAP16-27; SEQ ID NO:9, tPACAP22-27; SEQ ID NO:10, tPACAP9-21; SEQ ID NO:11, tPACAP8; SEQ ID NO:12, tPACAP15; and SEQ ID NO:13, tPACAP21). EC₅₀ values with respect to [Ca2+]i increases in FPRL1/RBL cells were then calculated. Sequential N-terminal truncations resulted in progressively lower efficacies, which suggest that this region contributes only partially to FPRL1 (SEQ ID NO:4) activation. However, none of the C-terminal-truncated PACAPs exhibited activity, indicating that the C-terminal sequences are critical for the activation of FPRL1 (SEQ ID NO:4). Interestingly, tPACAP9-27 (SEQ ID NO:7) was shown to partially activate FPRL1 (SEQ ID NO:4), despite the inability of tPACAP9-27 (SEQ ID NO:7) to activate PAC1, indicating that PACAP27 (SEQ ID NO:1) stimulates these FPRL1 (SEQ ID NO:4) and PAC1 in different ways. The binding affinity of tPACAPs to FPRL1 (SEQ ID NO:4) was also measured. As shown in FIG. 6B, this binding normally correlates with calcium increasing activity, but tPACAP9-27 (SEQ ID NO:7) exhibited almost the same binding affinity as PACAP27 (SEQ ID NO:1) (Kd=52.3+1.6 nM). These results suggest that the N-terminal region (1st to 8th) of PACAP27 (SEQ ID NO:1) is not associated with binding affinity, but rather that it contributes to full activation.

Based on an analysis of the VIP sequence (SEQ ID NO:3), which is similar to that of PACAP27 (SEQ ID NO:1), though it does not interact with FPRL1 (SEQ ID NO:4), several chimeric PACAPs (cPACAPs) were designed by substituting VIP (SEQ ID NO:3) amino acid residues (FIG. 6C) (SEQ ID NO:14, cPACAP24,25VIP; SEQ ID NO:15, cPACAP9VIP; SEQ ID NO:16, cPACAP13VIP; SEQ ID NO:17, cPACAP25VIP; and SEQ ID NO:18, cPACAP24VIP). Substitutions of the 24th (cPACAP24VIP; SEQ ID NO:18) or the 25th (cPACAP25VIP; SEQ ID NO:17) amino acids resulted in a pronounced loss of activity (FIG. 6D), and of binding affinity (Kd=2.1+0.13 M, Kd=2.0+0.17 M respectively), whereas substitutions of 13th (cPACAP13VIP; SEQ ID NO:16) or 9th (cPACAP9VIP; SEQ ID NO:15) had no effect on binding affinity (Kd=51.2+3.3 nM) (FIG. 6E). cPACAP24,25VIP (SEQ ID NO:14) had lowest binding affinity (Kd=8.7+0.75 M). Thus, it appears that C-terminal amino acid residues from 22 to 27 are primary contributors to binding and subsequent receptor activation, and that the 24th and 25th hydrophobic amino acid residues are major determinants. The central region from 9 to 21 seems to contribute only marginally to receptor binding and activation, and that the N-terminal region from 1 to 8 is required for full activation.

### Example 2

### Investigation of SAA Activities

2.1. Materials and Methods

2.1.1. Isolation and Culture of Synovial Fibroblasts and HUVECs

Fibroblast-like synoviocytes (FLS) were prepared from synovial samples obtained from patients suffering from RA and osteoarthritis (OA), all of whom were also undergoing total joint replacement surgery. The FLS were isolated from the synovial tissues in accordance with a previously described procedure (Cho, C. S., M. L. Cho, S. Y. Min, W. U. Kim, D. J. Min, S. S. Lee, S. H. Park, J. Choe, and H. Y. Kim. 2000. CD40 engagement on synovial fibroblast up-regulates production of vascular endothelial growth factor. J. Immunol. 164: 5055-5061).

In brief, fresh synovial tissues were minced into 2- to 3-mm pieces, then treated for 4 h with 4 mg/ml type I collagenase (Worthington Biochemical), and maintained in Dulbecco's Modified Eagle's Medium (DMEM) containing 10% FBS at 37° C. in an atmosphere containing 5% CO₂. The cells were used at 3 to 8 passages, during which time they evidenced a homogenous fibroblast population, and also exhibited a typical bipolar FLS configuration, as observed under inverse microscopy. MH7A cells, the immortalized synoviocytes that harbor the SV40 T antigen, were grown in DMEM supplemented with 10% FBS, as previously described (Miyazawa, K, A. Mori, and H. Okudaira. 1998. Establishment and characterization of a novel human rheumatoid fibroblast-like synoviocyte line, MH7A, immortalized with SV40T antigen. J. Biochem. 124: 1153-1162), and then employed in some of the experiments. Human umbilical vein endothelial cells (HUVECs) were isolated from fresh human umbilical cords via collagenase (Worthington Biochemical) digestion, and then maintained in 20% FBS-containing M-199 medium (Sigma, St. Louis, Mo.), as previously described. All HUVECs were used after no more than five passages.

2.1.2. Cell Proliferation Assay

The RA FLS, OA FLS, and HUVECs were plated onto 24-well culture dishes at a density of 2×10⁴ cells/well, and then permitted to attach overnight. After 24 h of serum starvation, the cells were treated for 72 h with a variety of mitogens. [³H]-thymidine (1 μCi) was added to each of the wells prior to the final 6 h of incubation. Cell growth was also evaluated via cell counts. Control and mitogen-treated cells were harvested by trypsinization, and the number of cells was determined with a hemocytometer, under ×100 magnification.

2.1.3. Apoptosis Assay

Synoviocyte apoptosis was induced via 3 days of serum deprivation, or by treating the cells for 12 h with either SNP (0.7 mM) or anti-Fas IgM (0.7 μg/ml) plus cyclohexamide (CHX; 1 μg/ml). The degree of apoptosis was then evaluated via MTT assay and ELISA for DNA fragmentation. In the MTT assay, FLS were seeded in 24-well culture plates at a density of 2×10⁴ cells/well. After 96 h of incubation with SAA (SEQ ID NO:19) or media alone, MTT solution was added to each of the wells, and then incubated for 2 additional hours. The reaction was halted via the removal of MTT. Thereafter, DMSO (200 μL) was added in order to solubilize the formazan crystals. The plates were then subjected to 5 minutes of gentle shaking in order to ensure that the crystals had dissolved completely, and the absorbance was read at 540 nm with a microplate reader. The cellular DNA fragmentation assay was conducted using an ELISA kit (Roche Applied Science), based on the quantitative sandwich ELISA principle, using two mouse monoclonal antibodies (Roche Applied Science) targeted against DNA and 5-bromo-2′-deoxyuridine (BrdU).

In brief, the BrdU-labeled DNA fragments of the samples were bound to the immobilized anti-DNA antibody, fixing it within the wells of a microtiter plate. The immune-complexed BrdU-labeled DNA fragments were then denatured and fixed to the surfaces of the plates via the application of microwave irradiation. In the final step, the anti-BrdU peroxidase conjugate was allowed to react with the BrdU that had been incorporated into the DNA. After the removal of the unbound peroxidase conjugates, the quantity of peroxidase bound within the immune complex was determined photometrically, using TMB as a substrate.

2.1.4. Generation and Transfection of Short Interfering RNA for FPRL1 Transcripts

In order to down-regulate the FPRL1 transcripts using short interfering RNA (siRNA), the following target sequences were used: ³⁰⁰AAU UCA CAU CGU GGU GGA CAU³²⁰ (SEQ ID NO:21) and ⁴⁰³AAC CAC CGC ACU GUG AGU CUG⁴²³ (SEQ ID NO:22). The results of a BLAST search of all siRNA sequences revealed no significant homology to any other sequences stored in the database. These two oligonucleotides yielded comparable results. MH7A immortalized synoviocytes were employed in the siRNA transfection procedure. These cells were transfected with a final concentration of 20 nM FPRL1 siRNA or luciferase siRNA, as a control, using LipofecAMINE reagent (Invitrogen) in accordance with the manufacturer's instructions. After 24 h of transfection, the cells were collected, after which the levels of FPRL1 expression were determined via reverse transcription-PCR. In brief, the total RNA from the transfected MH7A cells was isolated using a commercially-available TRI reagent (Molecular Research Center), in accordance with the manufacturer's instructions.

Complementary DNA (cDNA) was obtained by MMLV-RT (Promega) of 2 μg of total RNA with a random hexa-primer (Promega), after which PCR amplification was conducted for 27 cycles, each consisting of 30 seconds of denaturation at 95° C., 1 minute of annealing at 54° C., and 30 seconds of polymerization at 72° C. The following sense and antisense primers were employed for the detection of FPRL1 and β-actin (used as an internal control) for FPRL1, sense 5′-GAC CTT GGA TTC TTG CTC TAG TC-3′ (SEQ ID NO:23) and antisense 5′-TCA CAT TGC CTG TAA CTC AG-3′ (bp) (SEQ ID NO:24); for β-actin, sense 5′-TAC CTC ATG AAG ATC CTC A-3′ (SEQ ID NO: 25) and antisense 5′-TTC GTG GAT GCC ACA GGA C-3′ (bp) (SEQ ID NO:26). The PCR products were separated via electrophoresis through 1.5% agarose gel. The identities of the PCR products were verified by direct DNA sequencing.

2.1.5. Intracellular Ca²⁺ Measurement

The isolated FLS were incubated with Fluo3-AM working solution, containing 0.03% plutonic F-127 (the final concentration of Fluo3-AM was 20 μmol·L⁻¹) for 1 h at 37° C. After incubation, the cells were washed three times with normal or Na⁺- and K⁺-free Tyrode's solution, at 25° C. in order to remove the extracellular Fluo3-AM. Fluo3-AM fluorescence in the cells was elicited at 488 nm with a high-power Ar⁺ laser, and the emission bands were detected at 530 nm with a photomultiplier. The fluorescence signal was detected using a confocal laser scanning system (Biorad Lasersharp MRA2, Oxfordshire, UK), equipped with a Nikon E-600 Eclipse microscope. The fluorescence intensity (FI) was measured both prior to (FI₀) and after (FI) the addition of serum amyloid A (SAA) or phorbol-12-myristate-13-acetate (PMA) to either the normal the Na⁺- and K⁺-free Tyrode's solution. The change in [Ca²⁺]ᵢ, was expressed in terms of the (FI−FI₀)/FI₀ ratio. A total of 50-120 images were scanned in each cell.

2.1.6. Western Blot Analysis

RA FLS were incubated for 24 h in DMEM without FBS, and then SAA (SEQ ID NO:19) (3 μM) was added to RA-FLS for the indicated times. The treated RA-FLS was then washed twice in phosphate-buffered saline (PBS), dissolved in sample buffer (50 mM Tris-HCl, 100 mM NaCl, 0.1% sodium dodecyl sulfate (SDS), 1% NP-40, 50 mM NaF, 1 mM Na3VO4, 1 μg/ml aprotinin, 1 μg/ml pepstatin, and 1 μg/ml leupeptin), boiled, separated via SDS-polyacrylamide gel electrophoresis, and transferred to nitrocellulose membranes. After immunoblot analysis with phospho-ERK1/2 (Thr 202/Tyr 204), phospho-Akt (Ser 473), phospho-STAT3 (Tyr 705), Cylin D1, or Bcl-2 antibodies, the membranes were stripped and re-incubated with anti-Actin antibody in order to detect total protein amounts.

2.1.7. Wounding Migration and Tube Formation Assay

The wounding migration and tube formation activity of the HUVECs were measured as previously described (30, 31). In brief, HUVECs plated at confluence on 60-mm culture dishes were wounded with pipette tips, then treated with SAA (SEQ ID NO:19) (0-5 μg/ml), WKYMVm (SEQ ID NO:5) (10 nM), or VEGF (20 ng/ml) in M199 medium, supplemented with 1% serum and 1 mM thymidine. After 12 h of incubation, migration was quantitated via counts of the cells migrating beyond the reference line. For the tube formation assay, the HUVECs were seeded on a layer of previously polymerized Matrigel (BD Biosciences) with SAA (SEQ ID NO:19) (5 μg/ml), WKYMVm (SEQ ID NO:5) peptide (10 nM), a specific ligand for FPRL1 (32, 33) or VEGF (20 ng/ml). After 18 h of incubation, the cell morphology was visualized via phase-contrast microscopy and photographed.

2.1.8. Rat Aorta Ring Assay

Aortas from male Sprague-Dawley rats were cross-sectioned into rings, and mounted onto polymerized Matrigel dishes. Matrigel (150 μl) was then positioned on top and allowed to gel. After 7 days, the aortic rings, incubated with PBS, SAA (SEQ ID NO:19) (3 and 5 μg/ml), WKYMVm (SEQ ID NO:5) (10 nM), VEGF (20 ng/ml), or FBS (10%) were analyzed under an inverted microscope.

2.1.9. Mouse Matrigel Plug Assay

C57BL/6 mice (7 weeks of age) were given s.c. injections of 500 μl of Matrigel containing PBS, SAA (SEQ ID NO:19) (80 μg), or WKYMVm (SEQ ID NO:5) (1 μg). After 7 days, the skins of the mice were pulled back to expose the Matrigel plugs, which remained intact. After the noting and photographing of any quantitative differences, hemoglobin levels were measured via the Drabkin method, using a Drabkin reagent kit 525 (Sigma) for the quantitative assessment of blood vessel formation. The hemoglobin concentration was calculated from the parallel assay of a known amount of hemoglobin. The matrigel plugs were fixed in 4% formalin, embedded with paraffin, and stained using hematoxylin and eosin.

Statistical Analysis

All data are expressed as the means±standard deviation (SD) from several separate experiments. Statistical comparisons were conducted via Student's t-tests, and a P value of <0.05 was considered to be statistically significant.

2.2. Results

2.2.1. SAA Stimulates Synoviocyte Proliferation

Synovial hyperplasia is one of the hallmarks of RA pathology. Several studies have shown that RA FLS tend to divide at a more rapid rate than do synoviocytes obtained from normal or osteoarthritic joints. Therefore, it was attempted to determine whether SAA (SEQ ID NO:19) accelerates the proliferation of FLS acquired from both RA and OA patients, via [³H]-thymidine incorporation assays. When the FLS were stimulated with SAA (SEQ ID NO:19) (0.1-5 μM), the DNA synthesis activities of RA FLS and OA FLS increased in a dose-dependent fashion, with the maximal effect being detected at a SAA (SEQ ID NO:19) concentration of 5 μM (FIGS. 7A and 7C). Moreover, the numbers of RA FLS and OA FLS were also dose-dependently increased as the result of SAA (SEQ ID NO:19) treatment, and this effect was greater for the RA FLS than for the OA FLS (FIG. 7B). These results suggest that SAA (SEQ ID NO:19) is capable of stimulating the abnormal proliferation of FLS, particularly in joints afflicted with RA.

2.2.2. SAA Protects Rheumatoid Synoviocytes from Apoptotic Death

Previous investigations have demonstrated a lack of apoptotic cells in the synovial lining or the pannus in cases of RA FLS, and this anti-apoptotic characteristic appears to be required for synoviocyte hyperplasia in RA. Therefore, it was attempted to determine the effects of SAA (SEQ ID NO:19) on FLS apoptosis.

As is shown in FIGS. 8A and 8B, the treatment of RA FLS with SAA (SEQ ID NO:19) (0.1-5 μM) resulted in a dose-dependent inhibition of serum starvation-induced apoptosis, as determined by MTT assay and DNA fragmentation ELISA. The anti-apoptotic activity of SAA (SEQ ID NO:19) was shown to be more prominent in RA FLS than in OA FLS, a finding consistent with the currently-available data regarding SAA-induced synoviocyte proliferation (FIGS. 7A-7C). In RA-afflicted joints, the overproduction of nitric oxide (NO) as well as activated Fas signaling can induce apoptosis in the FLS. In order to simulate these conditions under in vitro conditions, sodium nitroprusside (SNP), a NO donor, or anti-Fas IgM Ab plus cycloheximide (CHX) was added to the cultured RA FLS. As had been expected, both SNP (0.7 mM) and anti-Fas (0.7 μg/ml) plus CHX (1 μg/ml) resulted in a high level of DNA fragmentation in RA FLS, but this was blocked almost completely by co-treatment with SAA (SEQ ID NO:19) (3 μM) (FIGS. 8C and 8D). Together, these data suggest that SAA (SEQ ID NO:19) is capable of rescuing RA FLS from apoptotic death in RA-afflicted joints.

2.2.3. FPRL1 (SEQ ID NO:4) Mediates SAA-Induced Proliferation and Survival of Synovial Fibroblasts

FPRL1 (SEQ ID NO:4) has been confidently identified as a receptor for SAA (SEQ ID NO:19). Therefore, in this example, the levels of FPRL1 (SEQ ID NO:4) expression in RA FLS and OA FLS were assessed. As is shown in FIG. 9A, all of the FLS expressed FPRL1 mRNA, and it was expressed significantly more abundantly in RA FLS than in OA FLS, thereby suggesting that RA FLS may respond in a more sensitive manner to FPRL1 ligation than OA FLS. Then, it was attempted to determine the role of FPRL1 (SEQ ID NO:4) in the SAA-induced proliferation and survival of FLS. Because FPRL1-blocking antibodies were commercially unavailable, WKYMVm (SEQ ID NO:5) peptide, a specific ligand for FPRL1, was used for the stimulation of FLS.

As is shown in FIG. 9B, the administration of the WKYMVm (SEQ ID NO:5) peptide induced a dose-dependent increase in the proliferation of RA FLS, but not OA FLS, while mitigating starvation-induced cell death. In order to verify that SAA activity is mediated by FPRL1 (SEQ ID NO:4) in the FLS, a blocking experiment was conducted by using short interfering RNA (siRNA) for FPRL1 transcripts. Two siRNA variants, with different sequences for human FPRL1, were designed, and were transiently transfected into MH7A immortalized FLS cells. As is shown in FIG. 9C, the levels of FPRL1 mRNA expression were reduced in the FLS transfected with FPRL1 siRNA, as compared to the levels observed in the siRNA-transfected or untransfected control cells. The knockdown of FPRL1 mRNA in the FLS resulted in the complete abrogation of SAA-induced cell proliferation and survival (FIG. 9D), whereas siRNA for luciferase, which was employed as the control siRNA, had no effect. Collectively, these results clearly indicate that FPRL1 (SEQ ID NO:4) is a major receptor which mediates SAA-induced proliferation and the survival of RA FLS.

2.2.4. SAA (SEQ ID NO:19) Ligation to FPRL1 (SEQ ID NO:4) Induces the Release of Intracellular Calcium

This experiment was conducted in order to evaluate the intracellular mechanisms inherent to effects of SAA (SEQ ID NO:19) on cellular proliferation and survival. Downstream events of FPRL1 activation are known to involve increases in intracellular Ca²⁺, which is involved in virtually all cellular processes, including cell survival, proliferation, and death. Accordingly, the influence of SAA (SEQ ID NO:19) on Ca²⁺ release in FLS was thought to warrant careful consideration.

Using a calcium-imaging system, it was determined that the addition of SAA (3 μM) to RA FLS induced a 2.3-fold increase in intracellular Ca²⁺, as compared to basal levels of Ca²⁺ (FIG. 10A). Moreover, the SAA-triggered release of Ca²⁺ was mimicked by the WKYMVm (SEQ ID NO:5) peptide (10 nM), and this increase was cancelled out by the pretreatment of cells with pertussis toxin (PTX) (100 ng/ml), an antagonist of the G-protein coupled receptor (GPCR) (FIG. 10B). These results indicate that SAA (SEQ ID NO:19) may evoke some rise in intracellular Ca²⁺ concentrations via FPRL1 (SEQ ID NO:4). It is noteworthy that RA FLS evidenced a higher degree of [Ca²⁺]ᵢ release than did OA FLS, when stimulated with SAA (SEQ ID NO:19), WKYMVm (SEQ ID NO:5), or phorbol myristate acetate (PMA) (100 nM) (FIGS. 10A and 10B). This shows that RA FLS harbors an intrinsic abnormality involving Ca²⁺ hyper-responsiveness to external stimuli, including SAA (SEQ ID NO:19), and this abnormality may be associated with cellular hyperactivation.

2.2.5. ERK and Akt Mediate the SAA-Induced Proliferation and Survival of Synoviocytes

Because ERK, Akt, and STAT3 activation are downstream targets of FPRL1 (SEQ ID NO:4), and are also critical for the proliferation and survival of several cell types, including RA FLS, in this example, it was attempted to determine whether SAA (SEQ ID NO:19) might induce the activation of ERK1/2, Akt, and STAT3 in RA FLS.

RA FLS was shown to respond to 3 μM of SAA (SEQ ID NO:19) with ERK1/2 and Akt phosphorylation, both of which proved detectable as early as 1 minute after stimulation, and peaked at 1 to 5 minutes afterward (FIG. 11A, upper panel). SAA (SEQ ID NO:19) was also implicated in a gradual increase in STAT3 activation, which began to occur 5 minutes after incubation, and evidenced maximal phosphorylation levels at 30 to 60 minutes (FIG. 11A, upper panel). The SAA-induced increases in ERK and Akt phosphorylation occurred in a time-dependent manner (FIG. 11A, middle panel). Moreover, both a dose and time-dependent activation of ERK and Akt were noted in RA FLS stimulated with various concentrations of WKYMVm (SEQ ID NO:5) (1 to 1000 nM), an agonistic peptide for FPRL1 (FIG. 11B). Therefore, it appears that SAA (SEQ ID NO:19) may trigger an increase in intracellular Ca²⁺ concentrations, as well as an increase in the activation of ERK1/2, Akt, and STAT3 via the FPRL1 receptor, thereby promoting the proliferation and survival of synoviocytes.

In order to address this hypothesis, a series of blocking experiments were conducted by using some pharmacological inhibitors of the above signaling molecules. As is shown in FIG. 5C, pretreatment of RA FLS with the GPCR inhibitor, PTX (100 ng/ml), the PLC inhibitor U73122 (1 μM), the MEK inhibitor PD98059 (50 μM), or the PI3K inhibitor LY294002 (50 μM) (6 h for PTX) for 1 h resulted in the almost complete blockage of the proliferative and anti-apoptotic activities of SAA. Collectively, our results show that the binding of SAA (SEQ ID NO:19) to FPRL1 (SEQ ID NO:4) facilitates the proliferation and survival of RA FLS via an increase in intracellular Ca²⁺ concentrations, as well as an enhancement of the activation of the ERK and Akt pathways.

The activation of the MAP kinases, ERK and Akt, contributes to the maintenance of mitochondrial integrity, via the upregulation of Bcl-2 expression. Based on the data regarding the survival advantage driven by SAA (SEQ ID NO:19), the effects of SAA (SEQ ID NO:19) on cyclin D1 expression, which induces the transition of cells from G1 arrest to the S phase, thereby leading to cell proliferation, were examined as well as the expression of Bcl-2, a representative anti-apoptotic molecule. When the RA-FLS were treated with SAA (SEQ ID NO:19) (3 μM) or WKYMVm (SEQ ID NO:5) (10 nM) for various times, cyclin D1 expression increased significantly, exhibiting peak values as early as 4 h after treatment (FIGS. 11A and 11B, lower panel). The expression of Bcl-2 was also gradually elevated 8 h after stimulation with SAA (SEQ ID NO:19) or WKYMVm (SEQ ID NO:5), and achieved peak levels between 12 to 24 h after stimulation (FIGS. 11A and 11B, lower panel). Collectively, these results suggest that SAA (SEQ ID NO:19) triggers the proliferation and survival of RA FLS, via the promotion of cyclin D1 and Bcl-2 expression.

2.2.6. SAA (SEQ ID NO:19) Increases Angiogenesis Via the Induction of Endothelial Proliferation, Migration, Tube Formation, and Sprouting Activity

It was finally attempted to determine whether SAA (SEQ ID NO:19) stimulates the proliferation of other types of FPRL1-harboring cells. As angiogenesis is considered to be a critical step in the progression of RA, and because human umbilical vein endothelial cells (HUVECs) express FPRL1 (SEQ ID NO:4) on the surfaces of the cells, the proliferation activity of SAA in experimental HUVECs was assessed. SAA (0.1 to 5 μM) induced DNA synthesis in the HUVECs in a dose-dependent manner, with the maximum effects occurring at 5 μM. These results were comparable to those generated in conjunction with the administration of 10 nM of WKYMVm (SEQ ID NO:5) peptide and 20 ng/ml of VEGF, a known mitogen in endothelial cells (FIG. 12A).

Furthermore, the HUVECs treated with SAA (SEQ ID NO:19) (5 μM) evidenced concentration-dependent increases in migration from the edge of the wound into the open area. The migratory activity of the HUVECs stimulated with SAA (SEQ ID NO:19) (5 μM), WKYMVm (SEQ ID NO:5) (10 nM), or VEGF (20 ng/ml) was approximately 3 times higher than that of the control cells (FIG. 12B). The effects of SAA (SEQ ID NO:19) on the morphological differentiation of endothelial cells in the tube formation assay were also examined. These findings indicated that the formation of elongated and robust tube-like structures was organized in a far superior fashion in the HUVECs treated with SAA (SEQ ID NO:19) (5 μM) than in the control HUVECs (FIG. 12C).

In order to confirm the angiogenic potential of the SAA, the sprouting of endothelial cells from aortic rings ex vivo and in vivo Matrigel plug angiogenesis trials were investigated in the presence of SAA (SEQ ID NO:19). As can be seen in FIG. 12D, the sprouting of endothelial cells increased as the result of SAA treatment, in a dose-dependent manner, whereas no sprouting cells were observed in the absence of SAA. Moreover, the in vivo exposed Matrigel mixtures harboring SAA (SEQ ID NO:19) (80 μg) or WKYMVm (SEQ ID NO:5) (1 μg) evidenced orange to red coloring, whereas the gels containing PBS retained their original white to amber coloring (FIG. 12E). In an attempt to quantify the angiogenesis in these samples, the hemoglobin contents of the Matrigel mixture gels were measured. The mean hemoglobin content of the SAA-treated Matrigels was 4.90±0.66 g/dL, whereas the hemoglobin content of the PBS-contained gels was 0.53±0.16 g/dL (P<0.05). The stained sections indicated that Matrigels containing the SAA (SEQ ID NO:19) or WKYMVm (SEQ ID NO:5) peptide had produced more vessels in the gels than had the Matrigel containing the PBS (FIG. 12F-H). These new vessels were filled with an abundance of intact red blood cells, indicating the formation of a functional vasculature within the Matrigels, and blood circulation in the newly-formed vessels resulting from the angiogenesis induced by treatment with SAA (SEQ ID NO:19) or the WKYMVm (SEQ ID NO:5) peptide. Collectively, these results appear to suggest that SAA (SEQ ID NO:19) has potent angiogenic activity, under in vitro, ex vivo, and in vivo conditions.

As aforementioned, the present invention provides a useful polymerized toner having a high chargeability and a good charge stability, by using a styrene-butadiene-styrene block copolymer as a pigment stabilizer, and by appropriately controlling a charge control agent with sulfonate group, to prevent a reduction of the chargeability due to the concentration of the pigment at the surface of the toner, thereby securing a high chargeability and a geed charge stability compared with the conventional polymerized toner.

1. A complex of PACAP27-FPRL1 having a regulatory effect on immune response.

2. The complex of PACAP27-FPRL1 according to claim 1, wherein the regulatory effect on immune response is to increase intracelluar calcium concentration, to stimulate extracellular signal-regulated kinase (ERK) phosphorylation, to up-regulate CD11b, or to induce chemotactic migration of neutrophil.

3. A composition for the use of treating or preventing diseases associated with immune response, containing i) an effective amount of inhibitor to inactivate the activity of PACAP27, FPRL1 or both of them, or to inhibit the binding of PACAP27 (SEQ ID NO:1) to FPRL1 (SEQ ID NO:4), or ii) inactivated PACAP27 (SEQ ID NO:1).

4. The composition according to claim 3, wherein the inhibitor is one or more selected from the group consisting of PACAP27 antagonists, the peptide WRWWWW (SEQ ID NO:6), GPCR (G protein-coupled receptor) inhibitors, and phospholipase C inhibitors.

5. The composition according to claim 3, wherein the inactivated PACAP27 (SEQ ID NO:1) has a modification at the amino acids “AA” positioned on 24^{th} and 25^{th} positions of C-terminus of PACAP27.

6. The composition according to claim 3, wherein the disease associated with immune response is resulted from increase of intracelluar calcium concentration, stimulation of extracellular signal-regulated kinase (ERK) phosphorylation, up-regulation of CD11b, or induction of chemotactic migration of neutrophil.

7. The composition according to claim 6, wherein the disease associated with immune response is an inflammatory condition.

8. A method of treating or preventing diseases associated with immune response by one or more method selected from the followings:
inactivating the activity of PACAP27, FPRL1, or both of them; and
inhibiting the binding of PACAP27 (SEQ ID NO:1) to FPRL1 (SEQ ID NO:4) to inhibit the formation of the PACAP27-FPRL1 complex.

9. The method according to claim 8, wherein an effective amount of inhibitor to inactivate the activity of PACAP27, FPRL1 or both of them, or inhibit the binding of PACAP27 (SEQ ID NO:1) to FPRL1 (SEQ ID NO:4) is administered to a patient in need, and the inhibitor is one or more selected from the group consisting of PACAP27 antagonists, the peptide WRWWWW (SEQ ID NO:6), GPCR (G protein-coupled receptor) inhibitors, and phospholipase C inhibitors.

10. The method according to claim 8, wherein PACAP27 (SEQ ID NO:1) is inactivated by a modification at the amino acids “AA” positioned on 24^{th} and 25^{th} positions of C-terminus of PACAP27.

11. The method according to claim 8, wherein the disease associated with immune response is resulted from increase of intracelluar calcium concentration, stimulation of extracellular signal-regulated kinase (ERK) phosphorylation, up-regulation of CD11b, or induction of chemotactic migration of neutrophil.

12. The method according to claim 11, wherein the disease associated with immune response is an inflammatory condition.

13. A target for developing drugs treating or preventing diseases associated with immune response containing the PACAP27-FPRL1 complex.

14. The target according to claim 13, wherein the disease associated with immune response is resulted from increase of intracelluar calcium concentration, stimulation of extracellular signal-regulated kinase (ERK) phosphorylation, up-regulation of CD11b, or induction of chemotactic migration of neutrophil.

15. The target according to claim 14, wherein the disease associated with immune response is an inflammatory condition.

16. A composition for the use of inhibiting synoviocyte hyperplasia and angiogenesis, containing an inhibitor to inactivate the activity of SAA, FPRL1, or both of them, or inhibit the binding of SAA (SEQ ID NO:19) to FPRL1 (SEQ ID NO:4).

17. The composition according to claim 16, wherein the inhibitor is one or more inhibitors selected from the group consisting of SAA antagonists, anti-FPRL1 antibodies for blocking of SAA (SEQ ID NO:19) binding to FPRL1 (SEQ ID NO:4), GPCR (G protein-coupled receptor) inhibitors, ERK inhibitors, or AKT inhibitors for blocking of the activation of intracellular signaling by SAA (SEQ ID NO:19).

18. A composition for the use of treating or preventing inflammatory diseases, containing an inhibitor to inactivate the activity of SAA and/or FPRL1, or inhibit the binding of SAA (SEQ ID NO:19) to FPRL1 (SEQ ID NO:4), wherein the composition has an inhibitory effect of synoviocyte hyperplasia and angiogenesis.

19. The composition according to claim 18, wherein the inhibitor is one or more inhibitors selected from the group consisting of SAA antagonists, anti-FPRL1 antibodies for blocking of SAA (SEQ ID NO:19) binding to FPRL1 (SEQ ID NO:4), GPCR (G protein-coupled receptor) inhibitors, ERK inhibitors, or AKT inhibitors for blocking of the activation of intracellular signaling by SAA.

20. The composition according to claim 18, wherein the inflammatory disease is selected from the group consisting of atherosclerosis, Alzheimer's disease, cancer, and Rheumatoid arthritis (RA).

21. A method of inhibiting synoviocyte hyperplasia and angiogenesis by inactivating the activity of SAA, FPRL1, or both of them, or inhibiting the binding of SAA (SEQ ID NO:19) to FPRL1 (SEQ ID NO:4) to inhibit the formation of the SAA-FPRL1 complex.

22. The method according to claim 21, wherein an effective amount of inhibitor to inactivate the activity of PACAP27, FPRL1 or both of them, or inhibit the binding of PACAP27 (SEQ ID NO:1) to FPRL1 (SEQ ID NO:4) is administered to a patient in need, and the inhibitor is one or more inhibitors selected from the group consisting of SAA antagonists, anti-FPRL1 antibodies for blocking of SAA (SEQ ID NO:19) binding to FPRL1 (SEQ ID NO:4), GPCR (G protein-coupled receptor) inhibitors, ERK inhibitors, or AKT inhibitors for blocking of the activation of intracellular signaling by SAA.

23. A method of treating or preventing inflammatory diseases by inactivating the activity of SAA, FPRL1, or both of them, or inhibiting the binding of SAA (SEQ ID NO:19) to FPRL1 (SEQ ID NO:4) to inhibit the formation of the SAA-FPRL1 complex.

24. The method according to claim 23, wherein an effective amount of inhibitor to inactivate the activity of PACAP27, FPRL1 or both of them, or inhibit the binding of PACAP27 (SEQ ID NO:1) to FPRL1 (SEQ ID NO:4) is administered to a patient in need, and the inhibitor is one or more inhibitors selected from the group consisting of SAA antagonists, anti-FPRL1 antibodies for blocking of SAA (SEQ ID NO:19) binding to FPRL1 (SEQ ID NO:4), GPCR (G protein-coupled receptor) inhibitors, ERK inhibitors, or AKT inhibitors for blocking of the activation of intracellular signaling by SAA.

25. The method according to claim 23, wherein the inflammatory disease is selected from the group consisting of atherosclerosis, Alzheimer's disease, cancer, and Rheumatoid arthritis (RA).

26. A target for developing drugs treating or preventing inflammatory diseases containing complex of SAA (SEQ ID NO:19) and FPRL1 (SEQ ID NO:4).

27. The target according to claim 26, wherein the inflammatory disease is selected from the group consisting of atherosclerosis, Alzheimer's disease, cancer, and Rheumatoid arthritis (RA).


SEQUENCE LISTING (26 sequences)

SEQ ID NO: 1 - protein, 27 aa, Homo sapiens
HSDGIFTDSYSRYRKQMAVKKYLAAVL

SEQ ID NO: 2 - protein, 38 aa, Homo sapiens
HSDGIFTDSYSRYRKQMAVKKYLAAVLGKRYKQRVKNK

SEQ ID NO: 3 - protein, 28 aa, Homo sapiens
HSDAVFTDNYTRLRKQMAVKKYLNSILN

SEQ ID NO: 4 - protein, 351 aa, Homo sapiens
METNFSTPLNEYEEVSYESAGYTVLRILPLVVLGVTFVLGVLGNGLVIWVAGFRMTRTVTTICYLNLALADFSFTATLPFLIVSMAMGEKWPFGWFLCKLIHIVVDINLFGSVFLIGFIALDRCICVLHPVWAQNHRTVSLAMKVIVGPWILALVLTLPVFLFLTTVTIPNGDTYCTFNFASWGGTPEERLKVAITMLTARGIIRFVIGFSLPMSIVAICYGLIAAKIHKKGMIKSSRPLRVLTAVVASFFICWFPFQLVALLGTVWLKEMLFYGKYKIIDILVNPTSSLAFFNSCLNPMLYVFVGQDFRERLIHSLPTSLERALSEDSAPTNDTAANSASPPAETELQAM

SEQ ID NO: 5 - protein, 6 aa, Homo sapiens
  MISC_FEATURE (6)..(6): Met at position 6 = D-met
WKYMVM

SEQ ID NO: 6 - protein, 6 aa, Homo sapiens
WRWWWW

SEQ ID NO: 7 - protein, 19 aa, Homo sapiens
SYSRYRKQMAVKKYLAAVL

SEQ ID NO: 8 - protein, 12 aa, Homo sapiens
QMAVKKYLAAVL

SEQ ID NO: 9 - protein, 6 aa, Homo sapiens
YLAAVL

SEQ ID NO: 10 - protein, 13 aa, Homo sapiens
SYSRYRKQMAVKK

SEQ ID NO: 11 - protein, 8 aa, Homo sapiens
HSDGIFTD

SEQ ID NO: 12 - protein, 15 aa, Homo sapiens
HSDGIFTDSYSRYRK

SEQ ID NO: 13 - protein, 21 aa, Homo sapiens
HSDGIFTDSYSRYRKQMAVKK

SEQ ID NO: 14 - protein, 27 aa, Homo sapiens
HSDGIFTDSYSRYRKQMAVKKYLNSVL

SEQ ID NO: 15 - protein, 27 aa, Homo sapiens
HSDGIFTDNYSRYRKQMAVKKYLAAVL

SEQ ID NO: 16 - protein, 27 aa, Homo sapiens
HSDGIFTDSYSRLRKQMAVKKYLAAVL

SEQ ID NO: 17 - protein, 27 aa, Homo sapiens
HSDGIFTDSYSRYRKQMAVKKYLASVL

SEQ ID NO: 18 - protein, 27 aa, Homo sapiens
HSDGIFTDSYSRYRKQMAVKKYLNAVL

SEQ ID NO: 19 - protein, 122 aa, Homo sapiens
MKLLTGLVFCSLVLGVSSRSFFSFLGEAFDGARDMWRAYSDMREANYIGSDKYFHARGNYDAAKRGPGGVWAAEAISDARENIQRFFGHGAEDSLADQAANEWGRSGKDPNHFRPAGLPEKY

SEQ ID NO: 20 - protein, 176 aa, Homo sapiens
MTMCSGARLALLVYGIIMHSSVYSSPAAAGLRFPGIRPEEEAYGEDGNPLPDFDGSEPPGAGSPASAPRAAAAWYRPAGRRDVAHGILNEAYRKVLDQLSAGKHLQSLVARGVGGSLGGGAGDDAEPLSKRHSDGIFTDSYSRYRKQMAVKKYLAAVLGKRYKQRVKNKGRRIAYL

SEQ ID NO: 21 - RNA, 21 nt, Homo sapiens
aauucacaucgugguggacau

SEQ ID NO: 22 - RNA, 21 nt, Homo sapiens
aaccaccgcacugugagucug

SEQ ID NO: 23 - DNA, 23 bp, Artificial Sequence
  Synthetic Primer
gaccttggattcttgctctagtc

SEQ ID NO: 24 - DNA, 20 bp, Artificial Sequence
  Synthetic Primer
tcacattgcctgtaactcag

SEQ ID NO: 25 - DNA, 19 bp, Artificial Sequence
  Synthetic Primer
tacctcatgaagatcctca

SEQ ID NO: 26 - DNA, 19 bp, Artificial Sequence
  Synthetic Primer
ttcgtggatgccacaggac